(19) United States
(12) Patent Application Publication
(10) Pub. No.: US 2024/0127957 A1
(43) Pub. Date: Apr. 18, 2024

(54) LOGIC-BASED TYPING OF MULTIPLE SCLEROSIS SUBJECTS BASED ON CODED DATA

(71) Applicants: GENENTECH, INC., South San Francisco, CA (US); HOFFMANN-LA ROCHE INC., Little Falls, NJ (US)

(72) Inventors: Shemra Rizzo, Durham, NC (US); Kelly Anne Zalocusky, Belfair, WA (US); Katherine Anne Belendiuk, Bozeman, MT (US); Nicole Gidaya Bonine, Maple Grove, MN (US); Yifeng Chia, Irvine, CA (US); Laura Gaetano, Basel (CH); Ryan William Gan, San Mateo, CA (US); Jumaah Ingram Goldberg, Greenville, SC (US); Xiaoming Jia, Berkely, CA (US)

(73) Assignees: GENENTECH, INC., South San Francisco, CA (US); HOFFMANN-LA ROCHE INC., Little Falls, NJ (US)

(21) Appl. No.: 18/506,808

(22) Filed: Nov. 10, 2023

Related U.S. Application Data
(63) Continuation of application No. PCT/US2022/027030, filed on Apr. 29, 2022.
(60) Provisional application No. 63/187,783, filed on May 12, 2021.

(51) Int. Cl. G16H 50/20; G16H 10/60; G16H 50/50

(52) U.S. Cl. CPC G16H 50/20; G16H 10/60; G16H 50/50

(57) ABSTRACT

Techniques disclosed herein relate to inferring a type of multiple sclerosis and determining whether to output an alert based on codes detected within noisy assessment data corresponding to a subject. The noisy assessment data includes content originating from a care provider that identifies a characteristic of the subject or of a treatment for the subject, and the subject has been diagnosed with multiple sclerosis. A temporal dynamic or distribution is determined based on instances of the code detection, and a modulation is determined based on the temporal dynamic or the distribution. It is determined whether an alert criterion is satisfied based on whether the modulation is above a threshold so as to represent noise or a predicted transition across types of multiple sclerosis. The inferred type of multiple sclerosis is output. When the alert criterion is satisfied, an alert is output as well.

### CROSS-REFERENCE TO RELATED APPLICATIONS

This application is a continuation of PCT/US2022/027030, filed on Apr. 29, 2022, which claims the benefit of and priority to U.S. Provisional Application No. 63/187,783, filed on May 12, 2021, each of which are hereby incorporated by reference in their entireties for all purposes.

### FIELD

Techniques disclosed herein relate to processing sets of codes (e.g., inconsistent codes) identified from noisy assessment data using logic-based operations to infer a type of multiple sclerosis that a subject had or has during a given time period. The type can be used to support: efficient and precise treatment selection, precise and controlled clinical-study enrollment, and/or expedient and consistent diagnoses.

### BACKGROUND

Multiple sclerosis (MS) is an immune-mediated disorder of the central nervous system (CNS). In MS, a subject's immune system is abnormally activated, traffics to the CNS, and causes injury to myelin sheaths that protect neurons (nerve cells) in the brain and/or spinal cord. Magnetic resonance imaging (MRI) can be used to detect “lesions,” or areas of injury resulting from abnormal immune activity (i.e. inflammation). If neurons remain unprotected (without a myelin sheath) for long enough, they may die (which appear as dark areas on some MRI images) and result in neurologic impairment and/or disability.

While inflammatory disease activity with clinical relapses underlies the beginning stages of multiple sclerosis for most subjects (relapsing multiple sclerosis, RMS), multiple sclerosis could also transition to (or, less commonly, could present initially as) a progressive disease course that resembles a neurodegenerative disease (primary or secondary progressive MS). In these circumstances, MRIs may not detect acute inflammatory disease activity, but rather show accelerated atrophy (shrinkage) of the subject's brain.

While there are stereotypical brain regions where disease activity occurs in multiple sclerosis, the disease may affect nearly all regions of the central nervous system. Thus, it is not surprising that the symptoms MS subjects experience are highly variable and can affect a variety of neurologic domains (e.g., cerebellar, brainstem, pyramidal, bladder and bowel, visual, mental).

Because multiple sclerosis has variable clinical presentation and lacks a single definitive diagnostic test, the diagnosis is made according to International Panel 2017 McDonald criteria, following exclusion of alternative causes. (See Thompson, A. J. “Diagnosis of multiple sclerosis: 2017 revisions of the McDonald criteria” Lancet Neurol. 2018 February; 17(2): 162-173. doi: 10.1016/S1474-4422(17)30470-2, which is hereby incorporated by reference in its entirety for all purposes). That is, a diagnosis of multiple sclerosis is provided after it has been determined that the subject's symptoms and test results cannot be explained by another disease (e.g., autoimmune, infectious, or other neurologic disorder). During clinical evaluation, a care provider may have varying levels of confidence in the diagnosis (“possible multiple sclerosis,” “probable multiple sclerosis,” “multiple sclerosis”). However, given the clinical heterogeneity, the diagnosis may be refined in the setting of (for example) changing care providers, new symptoms, or new clinical information.

Diagnosing a subject with multiple sclerosis is insufficient to determine which treatment may be the most effective for the subject (e.g., to suppress clinical relapses and reduce risk for disease progression). There are multiple types of multiple sclerosis, and the relative contribution of relapsing and progressive biology that underlie these types may differ, as does the efficacy of specific treatments. To date, there are over 20 FDA approved immunomodulatory disease modifying therapies in multiple sclerosis, each approved to treat one or more types of multiple sclerosis.

The most common types of multiple sclerosis are relapsing multiple sclerosis (RMS, also known as relapsing remitting multiple sclerosis or RRMS), secondary progressive multiple sclerosis (SPMS), and primary progressive multiple sclerosis (PPMS). Most subjects are initially diagnosed with relapsing multiple sclerosis, characterized by episodic “relapses” of acute inflammatory disease activity, often with accompanying clinical symptoms, followed by often incomplete remission. Years later, subjects with relapsing multiple sclerosis may transition to secondary progressive multiple sclerosis, a disease state characterized by progressive clinical worsening in the absence of clinical relapses. Subjects with relapsing multiple sclerosis may also experience insidious worsening between episodic relapses, suggesting that disease progression occurs along the spectrum of multiple sclerosis. Given the overlap in clinical characteristics, it may be challenging to identify the precise time that a subject transitions from relapsing multiple sclerosis to secondary progressive multiple sclerosis, in individuals who develop this progressive disease course.

Approximately 15% of multiple sclerosis subjects present with primary progressive multiple sclerosis, characterized by a progressive disease course from onset in the absence of clinical relapses. Like secondary progressive multiple sclerosis, primary progressive multiple sclerosis has features of neurodegeneration, and these sub-types are often collectively referred to as progressive multiple sclerosis (PMS). Given the incomplete recognition that multiple sclerosis may present with insidious worsening (see: Shah-Manek B., et al. “Rates and predictors of misdiagnosis in primary progressive multiple sclerosis (PPMS) in Europe and the United States” ECTRIMS. 2017 October; 200490; 0835, available at https://onlinelibrary.ectrims-congress.eu/ectrims/2017/ACTRIMS-ECTRIMS2017/200490/joanna.white.rates.and.predictors.of.misdiagnosis.in.primary.progressive.ht ml, which is hereby incorporated by reference in its entirety for all purposes), subjects with primary progressive multiple sclerosis may be initially (mis)diagnosed with another disease or another type of multiple sclerosis (most frequently relapsing multiple sclerosis).

Making an initial multiple sclerosis diagnosis, or changing a diagnosis from one type to another, could extend across multiple office visits and may delay identification of the right treatment. During these visits, a care provider may consider—in clinic notes—one or more types of multiple sclerosis and/or other diseases as part of the differential diagnosis.

Thus, even if medical records are available, it can be challenging to determine the subject correct type of multiple sclerosis. It may be even more challenging to map a subject's multiple sclerosis disease course from symptom onset to type transitions, which might be useful for clinical decision making.

Not being able to reliably discern a diagnosis of multiple sclerosis may impede care of the subject, clinical studies, and retrospective analyses. This includes selection of the best disease modifying therapy specific for the subject and multiple sclerosis type. Moreover, an entity performing a quality review (e.g., an entity administering the treatment) may inadvertently evaluate a treatment decision in view of an erroneous diagnosis. In addition, a subject may choose not to apply for a clinical study or may be denied for a clinical study based on an erroneous diagnosis or a mistaken impression of an actual diagnosis. As a result, clinical studies and/or retrospective analyses may also be improperly balanced across arms in a study.

Thus, there is a need to more accurately and consistently detect diagnoses of multiple diagnosis and its subtypes at a given point in time and/or retroactively.

### SUMMARY

In some embodiments, a computing system is provided that includes type inference logic, modulation determination logic, alert generation logic, an alert generator, and an output interface. The type inference logic selects a set of code instances within a defined time period. Each of the set of code instances indicates that a code of a set of codes was detected within noisy assessment data corresponding to a subject. The noisy assessment data includes content originating from a care provider that identifies a characteristic of the subject or of a treatment for the subject. The subject has been diagnosed with multiple sclerosis. The type inference logic further infers a type of multiple sclerosis that corresponds to the subject and a defined time period based on the set of code instances. The modulation determination logic determines a temporal dynamic or distribution based on the code instances detected within the noisy assessment data and determines a modulation based on the temporal dynamic or distribution. The alert generation logic determines whether an alert criterion is satisfied based on whether the modulation is above a threshold so as to represent noise or a predicted transition across types of multiple sclerosis. The alert generator generates an alert when the alert generation logic determines that the alert criterion is satisfied. The output interface outputs the inferred type of multiple sclerosis. The output interface further outputs the alert when the alert generation logic determines that the alert criterion is determined to be satisfied.

The type inference logic may further, in response to inferring that the type of multiple sclerosis corresponds to the subject and the defined time period: infer that the type of multiple sclerosis corresponds to the subject and another later time period that is subsequent to the defined time period.

Inferring that the type of multiple sclerosis corresponds to the subject and the defined time period may include: detecting that the subject was inferred to have had another type of multiple sclerosis during another time period that was before the defined time period; retrieving a type criterion associated with a previous inference of the other type of multiple sclerosis; determining, based on the set of code instances, that the type criterion is not satisfied; in response to determining that the type criterion is not satisfied, evaluating another type criterion using the set of code instances; determining that the other type criterion is satisfied; and determining that the type of multiple sclerosis is associated with the other type criterion.

Inferring that the type of multiple sclerosis corresponds to the subject and the defined time period may include: detecting that the subject was inferred to have had another type of multiple sclerosis during another time period that was before the defined time period; retrieving a type criterion associated with a previous inference of the other type of multiple sclerosis; determining, based on the set of code instances, that the type criterion is not satisfied; in response to determining that the type criterion is not satisfied, iteratively evaluating each of multiple other type criteria until one of the multiple other type criteria is satisfied; and determining that the type of multiple sclerosis is associated with the one of the multiple other type criteria that is satisfied.

The alert criterion may be configured to be satisfied when the modulation indicates that the subject transitioned from a particular type of multiple sclerosis to a different particular type of multiple sclerosis, and the transition may be inconsistent with a definition of the different particular type of multiple sclerosis.

The alert criterion may be configured to be satisfied when the set code instances include a particular code instance representing a particular type of multiple sclerosis and a term representing an observation that is inconsistent with the particular type of multiple sclerosis.

The inferred type of multiple sclerosis may be one of: relapsing-remitting multiple sclerosis, secondary progressive multiple sclerosis, primary progressive multiple sclerosis, or progressive relapsing multiple sclerosis.

The set of codes may include one or more codes representing a relapse, one or more codes representing progression, and one or more codes representing a type of multiple sclerosis.

The alert criterion may be configured such that a dissatisfaction of the alert criterion represents predicted disease stability.

Selecting the set of code instances within the defined time period may include detecting that, for each code instance in the set of code instances, a time stamp associated with the code instance is within the defined time period.

In some instances, a method is provided. The method includes selecting a set of code instances within a defined time period, wherein each of the set of code instances indicates that a code of a set of codes was detected within noisy assessment data corresponding to a subject, wherein the noisy assessment data includes content originating from a care provider that identifies a characteristic of the subject or of a treatment for the subject, and wherein the subject has been diagnosed with multiple sclerosis. The method includes inferring a type of multiple sclerosis that corresponds to the subject and the defined time period based on the set of code instances. The method also includes determining a temporal dynamic or distribution based on the set of code instances; determining a modulation based on the temporal dynamic or the distribution; and determining whether an alert criterion is satisfied based on whether the modulation is above a threshold so as to represent noise or a predicted transition across types of multiple sclerosis. The method further includes outputting the inferred type of multiple sclerosis; and when the alert criterion is satisfied: generating an alert; and outputting the alert.

The method may also include selecting a treatment for the subject based on the inferred type of multiple sclerosis; and providing the selected treatment to the subject.

The method may include determining that the subject is eligible for a clinical study based in part on the inferred type of multiple sclerosis; and enrolling the subject in the clinical study.

The method may include assigning the subject to a cohort of a clinical study based at least in part on the inferred type of multiple sclerosis.

In some embodiments, a method is provided that includes providing, by a user, an identifier of a subject and a request to a computing system for an inferred type of multiple sclerosis corresponding to the subject. Responsive to the request, the computing system selects a set of code instances within a defined time period, wherein each of the set of code instances indicates that a code of a set of codes was detected within noisy assessment data corresponding to the subject, wherein the noisy assessment data includes content originating from a care provider that identifies a characteristic of the subject or of a treatment for the subject, and wherein the subject has been diagnosed with multiple sclerosis. Responsive to the request, the computing system also infers the type of multiple sclerosis that corresponds to the subject and the defined time period based on the set of code instances; determines a temporal dynamic or distribution based on the set of code instances; and determines a modulation based on the temporal dynamic or the distribution. Responsive to the request, the computing system further determines that an alert criterion is satisfied based on whether the modulation is above a threshold so as to represent noise or a predicted transition across types of multiple sclerosis; generates an alert in response to the determination that the alert criterion is satisfied; and outputs the inferred type of multiple sclerosis and the alert. The method also includes receiving, by the user, the inferred type of multiple sclerosis and the alert on a display of the computer; and determining, by the user, a treatment based on the inferred type of multiple sclerosis.

In some embodiments, use of an inferred type of multiple sclerosis in the treatment of a subject is provided. The inferred type of multiple sclerosis is provided by a computing system performing a set of actions that includes: selecting a set of code instances within a defined time period, wherein each of the set of code instances indicates that a code of a set of codes was detected within noisy assessment data corresponding to a subject, wherein the noisy assessment data includes content originating from a care provider that identifies a characteristic of the subject or of a treatment for the subject, and wherein the subject has been diagnosed with multiple sclerosis; inferring a type of multiple sclerosis that corresponds to the subject and the defined time period based on the set of code instances; determining a temporal dynamic or distribution based on the set of code instances; determining a modulation based on the temporal dynamic or the distribution; determining whether an alert criterion is satisfied based on whether the modulation is above a threshold so as to represent noise or a predicted transition across types of multiple sclerosis; outputting the inferred type of multiple sclerosis; and when the alert criterion is satisfied: generating an alert; and outputting the alert.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawings will be provided to the Office upon request and the payment of the necessary fee. For a general understanding of the features of the disclosure, reference is made to the drawings. In the drawings, like reference numerals have been used throughout to identify identical elements.

The present disclosure is described in conjunction with the appended figures:

FIG. 1 shows an exemplary network 100 for inferring types of multiple sclerosis and potentially issuing alerts based on modulations with codes.

FIG. 2 shows exemplary distributions of across each of multiple types of multiple sclerosis (relapsing multiple sclerosis, secondary progressive multiple sclerosis, and primary progressive multiple sclerosis) to which code instances were mapped a given subject across 14 years.

FIG. 3 shows exemplary code event chains for twenty subjects (where each row corresponds to a subject).

FIG. 4 shows an exemplary interface that identifies code instances and inferred types of multiple sclerosis for two subjects. Each line corresponds to a given year (identified in the third column).

FIG. 5 shows a flowchart for a process of inferring multiple sclerosis types and/or conditionally outputting alerts based on noisy assessment data.

FIG. 6 shows representations of the noisy assessment data and a processed version thereof.

FIG. 7 shows the distribution of inferred types of multiple sclerosis across subjects (checkered bars).

FIGS. 8A-8D show distributions of ages of subjects having different types of multiple sclerosis at the time when the first codes were detected.

FIG. 8E shows statistics about the ages of the subjects corresponding to times associated with the first codes.

FIGS. 9A-9C show distributions of ages of subjects having different types of multiple sclerosis at a most recent time.

FIG. 9D shows statistics of distributions of ages of subjects having different types of multiple sclerosis.

FIGS. 10A and 10B show the percentage of the subjects having different types of multiple sclerosis who were male versus female.

FIG. 11 shows exemplary code event chains for twenty subjects who were inferred to have had relapsing multiple sclerosis at the year of first mention of relapsing multiple sclerosis.

FIG. 12 shows exemplary code event chains for twenty subjects who were inferred to have had relapsing multiple sclerosis at the year of first mention of secondary progressive multiple sclerosis.

FIG. 13 shows exemplary code event chains for twenty subjects who were inferred to have had relapsing multiple sclerosis at the year of first mention of primary progressive multiple sclerosis.

FIG. 14 shows exemplary code event chains for twenty subjects for whom an inferred type of multiple sclerosis was not identified based on codes.

FIG. 15 shows exemplary code event chains for twenty subjects who inferred to have had first had relapsing multiple sclerosis across a time period associated with one or more noisy assessment data elements and to have then had secondary progressive multiple sclerosis during a subsequent time period.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

Noisy assessment data is accessed that include content provided by one or more care providers that characterize a given subject with possible, probable, or confirmed multiple sclerosis. The noisy assessment data can include inconsistent content, in that a diagnosis conveyed or suggested within the noisy assessment data (e.g., pertaining to whether the subject has multiple sclerosis or a type of multiple sclerosis that the subject has) may be different. This inconsistency may be a result of (for example) the subject's multiple sclerosis, an erroneous initial diagnosis, differences in opinions across care providers, changes in scientific understanding of the disease, human error while preparing one or more records, and/or differences in formats or content types across noisy assessment data. Further, since multiple sclerosis is a chronic disease, the noisy assessment data may represent assessments performed over many decades. Thus, the utility of the noisy assessment data is extremely limited. A given care provider may review a most recent noisy assessment data set in preparation for or during an assessment of the subject, but a complete set of the noisy assessment data are rarely used by other parties or even assessed in aggregate by any single care provider.

In some embodiments, techniques are provided for using the noisy assessment data to infer which subtype of multiple sclerosis the subject had during a given time period and/or to further flag select noisy assessment data elements that convey potentially important or inconsistent information. More specifically, the noisy assessment data are processed to detect each instance where any of a set of codes appears (corresponding to a “code instance” or “instance of a code”). The noisy assessment data can include multiple instances of a single code and/or at least one instance of two or more of the set of codes. The noisy assessment data may further include codes, terms, and/or content that is not represented in the set of code. A time stamp (e.g., representing a date of a corresponding assessment) associated with the noisy assessment data set (e.g., indexed data set or data record) is detected. The codes and timestamps can be used to infer a type of multiple sclerosis that the subject had during a given time period.

In some instances, the code instances associated with time stamps within the given time period are inconsistent (e.g., corresponding to different types of multiple sclerosis). These circumstances may warrant particular flags to signify significant events in the subject's medical history and/or may benefit from additional review. To identify these instances, modulation associated with the code instances detected within the noisy assessment data are determined. For example, each code instance can be mapped to a potential type of multiple sclerosis, and the modulation associated with the code instances can include a distribution across the potential types of multiple sclerosis or a characteristic of the distribution. The characteristic of the distribution may include (for example) a percentage or quantity of the code instances that were mapped to a given type of multiple sclerosis. As another example, each code instance can be mapped to a potential type of multiple sclerosis, and the modulation associated with the code instances may represent a temporal dynamic across the potential types of multiple sclerosis to which the code instances were mapped. The temporal dynamic may be determined (for example) by computing a predicted type of multiple sclerosis in sliding windows and detecting each change in the predicted type of multiple sclerosis.

The inferred type of multiple sclerosis can be output. Further, it can be determined whether an alert criterion is satisfied based on the modulation associated with the code instances. For example, the alert criterion may be configured to be satisfied when a quantity or percentage of code instances associated with any potential type of multiple sclerosis exceeds a predefined time period or when a change in a predicted type of multiple sclerosis is detected across sliding windows. If it is determined that the alert criterion is satisfied, an alert can be generated and output.

The inferred type of multiple sclerosis and any alert may be associated with a recent time period, a past time period, or current time period. In some instances, multiple sets of noisy assessment data elements associated with the subject are processed, each corresponding to a different time period. An inferred type of multiple sclerosis can then be determined for each time period, and the alert criterion can be assessed for each time period.

The inferred type of multiple sclerosis and any alert may be output to (e.g., transmitted to or presented at) a computing system associated with a care provider, a coordinator of a clinical study, the subject, etc. The output may be used (e.g., by a care provider) to select a treatment to recommend or prescribe for the subject. In some instances, one or more treatment options that correspond to the inferred state are also output. For example, the modulation associated with the set of terms may represent a prediction that the subject's multiple sclerosis recently progressed from one sub-type to another sub-type, and the output may identify treatments approved for treating the other sub-type of multiple sclerosis.

The output may be used (e.g., by a coordinator of a clinical study) to determine whether the subject is eligible for a clinical study and/or to which arm of a clinical study to assign the subject. In some instances, the inferred state (and potentially one or more other inferred states and/or the modulation) are used to determine whether a criteria of a clinical study is satisfied, and the output includes an indication as to whether the criterion is satisfied. Potentially, the inferred state and/or the modulation are used to automatically assign the subject to a given arm in a study (e.g., based on the inferred or known states and/or modulations associated with other subjects in the study).

When the output includes an alert, the alert may include one or more data elements of the noisy assessment data or content therein. The alert may further represent the alert criterion was satisfied and may request input that conveys a type of multiple sclerosis that the subject had during part or all of the time period. Such input may correspond to (for example) a correction of content in at least one of the noisy assessment data or a confirmation (or overriding) of the inferred type of multiple sclerosis.

The output may include a timeline representing a history of the subject's multiple sclerosis disease. The timeline may represent a date of disease onset, initial diagnosis, and/or changes in diagnosis (e.g., representing true changes in types of multiple sclerosis or corrections of initial diagnoses).

### II. Definitions

As referred to herein, “noisy assessment data” refers to electronic data that includes content originating from a care provider that pertains to a subject. The content may identify a characteristic of the subject. The characteristic may include (for example): an observation of a capability or impairment of the subject; a symptom that the subject reported experiencing; and so on. The content may identify a result of a medical test; an interpretation of a medical test; a potential diagnosis; a diagnosis; a prescription; and so on. The noisy assessment data may be stored in a temporary data store or a permanent data store. The noisy assessment data may have been extracted from a noisy assessment record.

As used herein, a “noisy assessment record” refers to an electronic record from which noisy assessment data is or was extracted. The noisy assessment record may be or may include an electronic file. A single noisy assessment record may include content originating from a device of a care provider and pertaining to an assessment of a subject.

As used herein, a “code” refers to a set of characters, one or more words, and/or one or more images. The code may be (for example) a word, acronym, root word, abbreviation, or combination thereof. The code may include a selection, an image, a check, etc. that (for example) corresponds to a presented list item.

As used herein, a “code instance” or “instance of a code” refers to a given code having been included in or detected within noisy assessment data and/or within a noisy assessment record. Thus, a given subject may be associated with multiple code instances corresponding to a single code (e.g., representing that noisy assessment data associated with the subject repeatedly included or identified the given code). For example, a single noisy assessment record associated with the subject may have included a given code multiple times, and/or each of multiple noisy assessment records associated with the subject may have included a given code. Thus, noisy assessment data may indicate that the given code was detected multiple time (e.g., and identify one or more time stamps indicating when underlying assessments were performed). Each code instance or instance of a code may correspond to a particular code and a particular subject. Each code instance may correspond to a particular time stamp and/or a particular noisy assessment record. Each code instance or instance of a code may further correspond to a particular position within a noisy assessment record.

As used herein, a “time stamp” refers to a metric that represents a time at which an observation, assessment, sample collection, or medical test that underlies content in noisy assessment data was performed. The time stamp may be a date, a month-year combination, a year, a quarter, etc. For example, a noisy assessment record may include clinic notes representing an in-office assessment of a subject performed by a care provider on a given date. A time stamp of the noisy assessment record and of noisy assessment data derived from the noisy assessment record can be the date. As another example, a noisy assessment record can include a care provider's interpretations of a medical test (e.g., an MRI). A time stamp of the noisy assessment record and of noisy assessment data derived from the noisy assessment record can be the date on which the medical test was performed.

As used herein, a “type of multiple sclerosis” refers to a phenotype of medical sclerosis. For example, a type of multiple sclerosis may include relapsing multiple sclerosis, secondary progressive multiple sclerosis, primary progressive multiple sclerosis, progressive relapsing multiple sclerosis, clinically isolated syndrome, or pediatric multiple sclerosis.

As used herein, a “modulation” associated with a set of code instances or with a set of codes refers to a characterization of a temporal dynamic of the set of codes, a temporal dynamic of intermediate variables determined based on the set of codes, a distribution of the set of codes, or a distribution of intermediate variables determined based on the set of codes. For example, for each of multiple subsets of the set of codes, an intermediate variable that corresponds to an inferred type of multiple sclerosis can be generated based on the subset. Each of the multiple subsets may be associated with a distinct time stamp or time window relative to that associated with other subsets. Thus, a modulation may characterize whether and/or how the inferred types of multiple sclerosis are noisy (e.g., versus being consistent) or are changing over time. The change over time may be gradual or abrupt, and the modulation may characterize the speed of the change or may merely detect that a change is occurring or has occurred.

As used herein, an “alert” refers to information that is output. The alert may be output via (for example) transmission or display.

### III. Exemplary Network for Processing Noisy Assessment Data to Generate Inferred Types of Multiple Sclerosis and Alerts

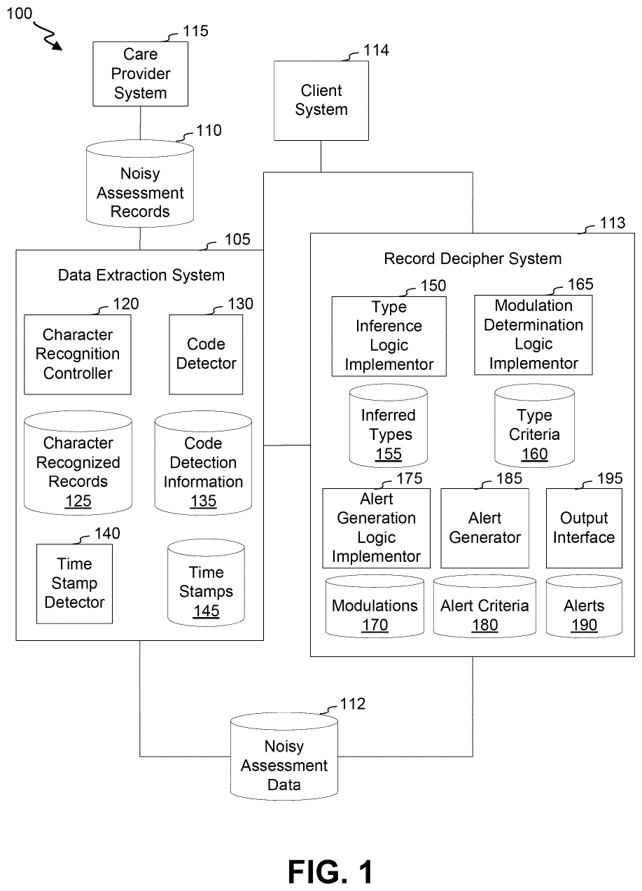

FIG. 1 shows an exemplary network 100 for inferring types of multiple sclerosis and potentially issuing alerts based on modulations associated with codes. Network 100 includes a data extraction system 105 that transforms a set of noisy assessment records 110 to noisy assessment data 112 (that includes select codes of interest and corresponding time stamps). Network 100 also includes a record decipher system 113 that uses noisy assessment data to infer which type of multiple sclerosis a given subject had or has and to determine whether to issue any alert.

Network 100 further includes a client system 114 that sends a request to data extraction system 105 to extract data and/or a request to record decipher system 113 to infer a type of multiple sclerosis that a subject has or had during a given time period (e.g., a current time period or a previous time period). In some instances, client system 114 sends a request to record decipher system 113, and record decipher system 113 thereafter sends a request to data extraction system 105 for noisy assessment data 112 that corresponds to a subject identified in the request. The request from client system 114 and/or the request from record decipher system 113 may include an identifier of the subject and potentially an identifier of the given time period.

Data extraction system 105 may then query a remote or local data store using the identifier(s) to retrieve the set of noisy assessment records 110. In some instances, the request from client system 114 includes or is accompanied by the set of noisy assessment records 110.

III.A. Noisy Assessment Records

Each of the set of noisy assessment records 110 can include content that characterize health of a subject who has been identified as possibly or probably having multiple sclerosis or who has been diagnosed with multiple sclerosis. The content can include an assessment of a care provider (e.g., neurologist, radiologist, physician, or nurse practitioner). Thus, each noisy assessment record 110 may have been generated at a care provider system 115 or may be generated based on information received from care provider system 115. For example, a noisy assessment record 110 may include a file that includes clinician notes that were input (e.g., via text or using a camera or scanner) at care provider system 115 and transmitted to a central server. It will be appreciated that the set of noisy assessment records 110 may have been generated based on input from multiple care providers (e.g., with one or more noisy assessment records 110 including content from one care provider and one or more other noisy assessment records 110 including content from another care provider).

The content may include: a first-person observation of the subject (e.g., posture, engagement, coherency, etc.); a first-person observation as to how well a subject performed one or more tasks (e.g., walking up and down a hallway, standing on one foot, sensing a vibration or poke, performing a fine-motor task, etc.); a symptom reported by the subject (e.g., tingling, numbness, pain, memory difficulties, etc.); and/or an interpretation of a medical test (e.g., whether a magnetic resonance image includes an enhancing lesion; how many lesions are in a recent magnetic resonance image as compared to a previous image; a degree of brain atrophy as determined based on time-separated magnetic resonance images; and/or where—within a subject's brain or spinal cord—various lesions are present). The assessment may indicate that the observation, symptom, and/or interpretation of the medical test is consistent with multiple sclerosis (or a specific type of multiple sclerosis).

The content may indicate that the care provider is contemplating diagnosing the subject with multiple sclerosis (or a type of multiple sclerosis), is diagnosing the subject with multiple sclerosis (or a type of multiple sclerosis), or is changing a diagnosis of the subject (e.g., from one type of multiple sclerosis to another type). The content may indicate that the subject was previously diagnosed with multiple sclerosis (or a particular type of multiple sclerosis) by the care provider or another care provider. The content may include an indication that the care provider has determined that or is postulating that the subject has experienced or is experiencing a relapse and/or that the subject's multiple sclerosis is worsening.

III.A.1. Types of Multiple Sclerosis

The content may indicate that a care provider identified a possible diagnosis, a probable diagnosis, or a diagnosis of a particular type of medical sclerosis. The type of multiple sclerosis can include (for example) relapsing-remitting multiple sclerosis, secondary progressive multiple sclerosis, primary progressive multiple sclerosis, progressive relapsing multiple sclerosis, or clinically isolated syndrome. Alternatively, the type of multiple sclerosis may include multiple sclerosis with or without ongoing worsening.

III.A.1.a. Relapsing Multiple Sclerosis (RMS)

In 85-90% of cases, multiple sclerosis first presents as a relapsing multiple sclerosis (RMS) type (also referred to as relapsing remitting multiple sclerosis (RRMS)). RMS subjects experience discrete exacerbations (or “relapses”) during which new neurological symptoms appear or during which old symptoms worsen. The prominent mechanism hypothesis is that exacerbations are a result of an autoimmune cascade during which autoreactive leukocytes traverse the blood-brain barrier from the periphery and attack the myelin protective layers on neuron projections in the central nervous system. The autoimmune cascade is thought to predominantly involve T cells but to also involve some B cells (e.g., to recruit macrophages, activate the complement pathway and/or produce costimulatory molecules that influence T cell differentiation). Another theory is that the attack is performed by myelin-specific T cells with malfunctioning immunoregulatory mechanisms.

Each exacerbation may result in one or more physiological symptoms and/or one or more new lesions. A physiological symptom may be a symptom associated with one of eight functional systems. Functional systems and select associated symptoms include the pyramidal system (symptoms: muscle weakness or difficulty moving limbs); cerebellar system (balance problems or tremor); brainstem system (difficulties with speech or swallowing); sensory system (numbness or tingling); bowel or bladder system (incontinence); visual system (blurriness or blindness); cerebral system (deficits in memory, multi-tasking or thinking); and other. Between exacerbations, symptoms may partly or completely disappear. Full recovery from relapses is more likely when the relapse occurred closer to a diagnosis date (as compared to later time periods).

Thus, a noisy assessment record 110 may identify a possible, probable, or confirmed diagnosis of relapsing multiple sclerosis if a care provider observes discrete incidences of new symptoms (potentially being separated by partial or complete symptom relief) and/or discrete and time-separated incidences of new lesions (e.g., inflammatory lesions.

III.A.1.b. Active or Inactive Secondary Progressive Multiple Sclerosis SPMS)

If left untreated, most subjects with relapsing multiple sclerosis will transition to secondary progressive multiple sclerosis (SPMS) within 25 years. There is no general agreement in the medical community as to what precise indicators mark a transition from relapsing multiple sclerosis to secondary progressive multiple sclerosis.

Secondary progressive multiple sclerosis subjects who also continue to experience symptomatic relapses and inflammation are deemed to have active secondary progressive multiple sclerosis (some members of the clinical community consider this as RMS), while those without detectable disease activity are characterized as having inactive secondary progressive multiple sclerosis. However, regardless, these subjects typically experience gradual and generally monotonic decline in function as a result of nerve damage or loss. Stated differently, the decline observed in secondary progressive multiple sclerosis subjects is generally understood to be primarily due to indolent progressive injury, not due to acute inflammatory disease activity.

A noisy assessment record 110 may identify a possible, probable, or confirmed diagnosis of secondary progressive multiple sclerosis if a care provider observes that a subject who was previously diagnosed with relapsing multiple sclerosis or who has a history consistent with relapsing multiple sclerosis is exhibiting progressive worsening of symptoms (e.g., independent of discrete relapses) and/or is becoming increasingly disabled (e.g., using a walker or wheelchair).

III.A.1.c. Primary Progressive Multiple Sclerosis (PPMS)

Some subjects with multiple sclerosis are never diagnosed with the relapsing type of multiple sclerosis and instead are initially diagnosed with Primary Progressive Multiple Sclerosis (PPMS). As with secondary progressive multiple sclerosis, these subjects generally experience gradual and sustained worsening of symptoms and brain injury. With regard to both secondary progressive multiple sclerosis and primary progressive multiple sclerosis, the gradual function loss may occur over a time period of months to years, making it difficult to detect. Most people with primary progressive multiple initially present with motor symptoms (while sensory and/or optical symptoms are more prevalent for relapsing multiple sclerosis).

A noisy assessment record 110 may identify a possible, probable, or confirmed diagnosis of primary progressive multiple sclerosis diagnosis if a care provider determines that a subject meets diagnostic criteria for MS, and is experiencing functional-system impairment that occurs gradually and independently of discrete relapses (such that the gradual worsening is not a result of any residual impairment triggered by relapses). The possible, probable, or confirmed diagnosis may further be restricted to instances where the care provide does not believe that the subject previously experienced relapsing multiple sclerosis.

A noisy assessment record 110 may also or alternatively identify primary progressive multiple sclerosis as a possible, probable, or confirmed diagnosis if a care provider observes that the subject is experiencing symptomatic relapses and/or enhancing lesions after a diagnosis of progressive multiple sclerosis (e.g., after a diagnosis of primary progressive multiple sclerosis). While these circumstances may have historically led to a diagnosis of Progressive-Relapsing sclerosis, more recently, primary progressive multiple sclerosis is an accepted diagnosis.

III.A.1.d. Clinically Isolated Syndrome (CIS)

Even though, Clinically Isolated Syndrome (CIS) is considered a type of multiple sclerosis, Clinically Isolated Syndrome is actually distinct from multiple sclerosis. A noisy assessment record 110 may identify a possible, probable, or confirmed diagnosis of Clinically Isolated Syndrome if a care provider observes that a subject experienced a first neurological symptom for at least 24 hours that is isolated in time and not due to another medical condition (e.g., stroke or Lyme's disease). If the subject recalls one or more other prior neurological symptoms or if an MRI identifies old (non-enhancing) lesions, the subject is to be diagnosed with multiple sclerosis, not Clinically Isolated Syndrome.

Subjects with Clinically Isolated Syndrome may, or may not, subsequently experience additional symptoms and receive a multiple-sclerosis diagnosis. Clinically Isolated Syndrome subjects may receive select multiple-sclerosis medications, which may reduce the probability that or extend a time at which the subject is diagnosed with multiple sclerosis.

III.A.1.e. Multiple Sclerosis with or without Ongoing Worsening

An approach that is currently being considered for classifying types of multiple sclerosis is to replace current definitions for types of multiple sclerosis (e.g., corresponding to types described in Sections III.A.1.a-d.) with two mutually exclusive types of multiple sclerosis: multiple sclerosis with or without active worsening. This approach would allow the classification to be made based on current or recent data without needing to consider whether the subtype of multiple sclerosis is known, in the setting of incomplete historical information. Multiple sclerosis with ongoing worsening may include MS subjects (of any subtype) who experience clinical or radiographic disease worsening (e.g., gradual or stepwise degradation in functional abilities, accelerated brain atrophy, accumulation of black holes), but when it is unclear whether a subject's multiple sclerosis presented with or without relapses. Multiple sclerosis without ongoing worsening may include MS subjects (of any subtype) who do not experience recent clinical or radiographic worsening, either related to acute disease activity or progressive indolent injury, but when it is unclear whether a subject's multiple sclerosis presented with or without relapses.

III.A.2. Medical Tests Potentially Informative for Multiple Sclerosis Diagnosis

A noisy assessment record 110 can include results from one or more medical tests and/or interpretation of results from one or more medical tests. The results and/or interpretation may be informative of a possible, probable, or confirmed diagnosis.

III.A.2.a. Magnetic Resonance Imaging

A noisy assessment record 110 may include an image produced by a magnetic Resonance Imaging (MRI) machine or interpretations thereof. An MRI machine includes large magnets that generate strong magnetic fields. These fields cause protons in the body to align with the field. A resonance-frequency radiofrequency (RF) field causes the protons to spin out of equilibrium against the magnetic field. When the RF field turns off, the protons release energy while realigning with the magnetic field. An MRI machine includes a receiving coil to measure this energy release. Different types of biological structures will result in different energy-release profiles (e.g., identifying a time elapsed to return to an equilibrium state). Two operation settings include a repetition time and an echo time. The repetition time is the time between successive RF pulses. Long repetition times enable all protons to realign with the magnetic field before a next pulse, whereas short repetition times result in many protons only partly re-aligning. The echo time indicates when signals produced by the protons are measured. Longer echo times make it more likely that protons in gray and white matter will go out of phase, which can result in longer signals. Meanwhile, fluids are less sensitive in this respect, so their signals will remain stronger.

MRI images can include T1-weighted images (T1 images), T2-weighted images (T2 images) or FLAIR images. T1 images are produced in response to short echo times (e.g., 14 ms) and short repetition times (e.g., 500 ms). In T1 images, white matter (e.g., axons) is light, gray matter (e.g., nerve cell bodies and dendrites) is gray, the spinal cord is gray, cerebrospinal fluid (CSF) is dark, and inflammation is dark. Black holes will appear in T1 images as hypointense (dark) areas. When a contrast agent (e.g., gadolinium) is administered to a subject, it may pass through the blood-brain barrier only if this barrier had been recently disrupted and can leak into the recently formed lesions. A T1 image will then depict these lesions as bright areas.

T2 images are produced in response to long echo times (e.g., 90 ms) and long repetition times (e.g., 4000 ms). In T2 images, white matter is dark gray, gray matter is light gray, the spinal cord is light gray, CSF is bright, and inflammation is bright. T2 images may thus be used to detect new and old lesions (which will appear bright). FLAIR images are produced in response to even longer echo times (e.g., 114 ms) and even longer repetition times (e.g., 9000 ms). In FLAIR images, white matter is dark gray, gray matter is light gray, the spinal cord is light gray, CSF is dark, and inflammation is bright. FLAIR images allow better visibility of lesions adjacent to the CSF as compared to T2 images.

Thus, a noisy assessment record 110 may identify how may lesions were detected in T1, T2 and/or FLAIR lesions. A location (e.g., brain region), size, and/or lesion type may further be identified for each lesion. Noisy assessment record 110 may further identify how depictions each of one more lesions have changed relative to a previous MRI (e.g., whether and/or how the lesion has grown, stopped enhancing, or shrunk).

MRI images of the brain and/or spinal cord can be used to facilitate a diagnosis of multiple sclerosis, to facilitate characterizing MS disease activity and/or MRI worsening in a subject, to facilitate characterizing a degree to which a treatment is effectively treating a subject, and/or to facilitate determining whether to change a treatment strategy for a subject. The number, location, size and shape of the lesions, and change in lesion volumes may inform one or more of these characterizations.

MRI analysis frequently involves determining whether MRI images depict any contrast-enhancing lesions, which indicate whether the subject's multiple sclerosis was recently active. T2-weighted lesions are also frequently used to measure total lesion volume. T2 disease-burden metrics measured early in the disease process (e.g., in Clinically Isolated Syndrome or relapsing multiple sclerosis) can provide information about risk for disease progression and/or long term disability. Noisy assessment record 110 may identify which (if any) lesions are enhanced, how many lesions are enhanced, and/or lesion burden.

Further, noisy assessment record 110 may include an assessment of MRI results that identifies a cross-sectional area (e.g., at a segment of the spinal cord) or volume (e.g., of the brain), which can be used to estimate an extent of atrophy in a subject. The atrophy may be estimated by comparing area/volume metrics generated based on a recent MRI image of the subject to those generated based on an older MRI image of the subject. The atrophy may further be estimated by comparing area/volume metrics generated based on a recent MRI image of the subject to statistics generated based on a comparable population. Atrophy statistics may be used to inform a care provider's determination as to whether a subject has possible, probable, or confirmed multiple sclerosis and/or the rate of worsening due to multiple sclerosis.

As further described below, the presence of lesions in multiple anatomical areas and/or time-separated appearance of lesions is required for the diagnosis of multiple sclerosis, and is useful for distinguishing between likely, probably, and definitive MS. Time-separated appearance of lesions may be detected by comparing MRIs collected at different time points or by detecting that at least one lesion is contrast enhanced (indicating recent inflammation) and that at least one lesion is not contrast enhanced. Noisy assessment record 110 may characterize a time-separated appearance of a lesion and/or may indicate that a given time-separated appearance of a lesion satisfies International Panel diagnostic criteria for multiple sclerosis, and to establish the level of confidence in such diagnosis.

III.A.2.b. Cerebrospinal Fluid Analysis

Noisy assessment record 110 may identify a result of an analysis of a subject's cerebrospinal fluid (CSF) and/or may indicate whether the result is consistent with multiple sclerosis. CSF of most subjects with multiple sclerosis (and of many subjects with other inflammatory medical conditions) include relatively high concentrations of intrathecal Immunoglobulin G (IgG) as compared to the general population. Elevated CSF IgG levels compared to serum (derived from blood) levels, often reported as IgG index, is observed in multiple sclerosis but is not part of established International Panel diagnostic criteria. Oligoclonal bands (OCBs), or unique IgGs found in CSF but not in serum, is a part of the established International Panel criteria used for diagnosis of MS. Lastly, CSF white blood cell (WBC) count, red blood cell (RBC), protein concentration, and other laboratory test may help confirm or exclude the diagnosis of multiple sclerosis in the right clinical setting.

Thus, analysis of CSF is frequently performed during a diagnostic stage of multiple sclerosis. CSF is collected from a subject via a lumbar puncture. One or more laboratory tests (e.g., protein electrophoresis, Western blot, or a combination of isoelectric focusing and silver staining) are performed to detect proteins in each of a CSF sample and potentially in a serum sample (e.g., a diluted serum sample).

The presence of 2 or greater OCBs in the CSF supports a diagnosis of multiple sclerosis, and serves as a predictive biomarker for the risk of developing clinically definite MS in subjects with CIS (individuals experiencing a single relapse who do not yet meet diagnostic criteria for multiple sclerosis).

An IgG CSF Index is defined as the ratio of IgG relative to albumin in the CSF as compared to the ratio of IgG relative to albumin in serum. Elevated IgG index indicates that the central nervous system is producing IgG. The IgG CSF Index can also be used to generate an IgG synthesis rate.

III.A.2.c. Visually Evoked Potentials

Noisy assessment record 110 may characterize a subject's visually evoked potentials and/or may indicate whether the subject's visually evoked potentials are consistent with multiple sclerosis, specifically whether multiple sclerosis affects the subject's optic tracts.

Myelination of axons increases the speed at which action potentials move along the axons. Thus, if the myelin coasting is attacked via an inflammatory multiple sclerosis attack, the conductance of nerve impulses may subsequently be slowed. One test that can be informative in the diagnosis of multiple sclerosis is evoking action potentials and measuring the speed of their transmission. Typically, for this assessment, potentials are evoked by presenting a particular visual stimulus (e.g., a binary, dynamic checkerboard) while brain signals (e.g., in the occipital cortex) are non-invasively recorded (e.g., via electroencephalography). However, auditory or somatosensory stimuli may alternatively be used. The evoked potential includes two negative peaks and one positive peak (between the two negative peaks). The magnitude and/or time of each of these peaks can be informative of diagnosis and/or prognosis of multiple sclerosis. For example, multiple sclerosis may cause inflammatory injury to the optic nerves (optic neuritis) that can result in loss of the positive peak and/or highly attenuated responses.

III.A.3. Criteria Used for Multiple Sclerosis Diagnosis

Noisy assessment record 110 may indicate that a care provider has determined that the subject has a possible, probable, or confirmed diagnosis of multiple sclerosis. Noisy assessment record 110 may further provide support for the possible, probable, or confirmed diagnosis.

There is no single test or single assessment that reliably determines whether a subject has multiple sclerosis. In some instances, a test result (e.g., MRI images or CSF result) may be required to confirm a diagnosis of multiple sclerosis, but that test result may not be sufficient across subjects and circumstances.

Many care providers characterize the diagnosis of multiple sclerosis as a diagnosis of exclusion, in that the clinician is tasked with prescribing tests and performing analyses that would rule out potential other causes for observed abnormalities. For example, neurological symptoms and/or MRI results may be explained by infectious disease (e.g., Lyme's disease), spinal cord compression, vitamin B12 deficiency or non-MS inflammatory de-myelinating disease (e.g., sarcoidosis, systemic lupus erythematosus, neuromyelitis optical or acute disseminated encephalomyelitis). If no such alternative explanations can be identified, a diagnosis of multiple sclerosis made, following a clinical evaluation that also meets International Panel diagnostic criteria.

A diagnosis of exclusion, in many cases, may result in a delayed diagnosis and treatment. While MS subjects await testing, test results, and clinical assessments, it is possible that their disease continues to worsen. Various protocols and disease characterizations have attempted to take such situations into consideration. For example, the diagnosis of multiple sclerosis in some cases does not depend on ruling out all potential alternative causes (although some people may then discover—years after their multiple-sclerosis diagnosis—that they, in fact, have a different medical condition); further, the establishment of Clinically Isolated Syndrome (CIS), and/or diagnosis of clinically definite MS following positive CSF testing in subjects with CIS, has allowed physicians to prescribe approved disease-modifying therapies that reduce relapses and/or the risk for progression, in an expedited fashion. (See Thompson, A. J. “Diagnosis of multiple sclerosis: 2017 revisions of the McDonald criteria” Lancet Neurol. 2018 February; 17(2): 162-173. doi: 10.1016/S1474-4422(17)30470-2, which is hereby incorporated by reference in its entirety for all purposes.)

III.A.3.a. McDonald Criteria

A prominent criteria for diagnosing multiple sclerosis is the 2017 Revisions of the McDonald Criteria. (See Thompson, A. J. “Diagnosis of multiple sclerosis: 2017 revisions of the McDonald criteria” Lancet Neurol. 2018 February; 17(2): 162-173. doi: 10.1016/S1474-4422(17)30470-2, which is hereby incorporated by reference in its entirety for all purposes.) An underpinning of this criteria is that the physician is to generally have evidence of separation in space and time to diagnosis a subject with multiple sclerosis. Separation in space may correspond to symptoms affecting different parts of the body or different functional systems or lesions present in different parts of the central nervous system. Separation in time may correspond to symptoms and/or lesions appearing at different times, which may be detectable via multiple assessments (e.g., multiple MRIs or multiple clinical/symptomatic assessments) performed at different times or by detecting some contrast-enhancing lesions (e.g., indicating recent inflammation) and some non-enhanced lesions (e.g., indicating that they were not recently formed).

The McDonald criteria has emerged as a standard frequently used for diagnosis with multiple sclerosis. Under this criteria, any of the following circumstances results in a multiple sclerosis diagnosis, and a multiple sclerosis diagnosis is not to be otherwise made.
Two or more relapses and symptomatic clinical evidence of two or more lesions;
Two or more relapses, clinical evidence of a lesion in the central nervous system, and one or more lesions typical of multiple sclerosis;
Two or more relapses, clinical evidence of a lesion in the central nervous system, and another relapse indicating injury to another part of the central nervous system;
One relapse, clinical evidence of two or more lesions (e.g., impairment in two or more functional systems or two or more parts of the body); and disparate immune-system activity as evidenced by oligoclonal bands as identified based on multiple time-separated spinal taps;
One relapse, clinical evidence of two or more lesions (e.g., impairment in two or more functional systems or two or more parts of the body); and MRI evidence of a new lesion since a previous scan;
One relapse, clinical evidence of two or more lesions (e.g., impairment in two or more functional systems or two or more parts of the body); and a further symptomatic relapse;
One relapse, clinical evidence of one lesion, one or more MRI lesions typical of multiple sclerosis;
One relapse, clinical evidence of one lesion, another relapse showing activity in a different part of the central nervous system as indicated by oligoclonal bands, an MRI depicting a new lesion since a previous scan or a new relapse;
One relapse, clinical evidence of one lesion, one or more MRI lesions typical of multiple sclerosis; or
Gradual progression of neurological symptoms typical of multiple sclerosis for one year plus any two of: at least one brain lesion typical of multiple sclerosis, two or more lesions in the spinal cord, or oligoclonal bands in the spinal fluid.

III.A.4. Assessments of MS Disease Activity and Disease Progression

After a subject is diagnosed with multiple sclerosis, periodic appointments with a care provider (e.g., a neurologist) are typically made to assess various functions and convey current or recent symptoms. In some instances, these appointments are scheduled at regular intervals (e.g., every 6 months). In some instances, a subject requests one or more appointments to discuss new symptomatic exacerbations, medication side effects, etc. Noisy assessment record 110 may identify information shared by the subject, observations made by the care provider, and/or recommendations from the care provider conveyed during an appointment.

Care providers frequently also recommend that subjects periodically receive MRIs. Again, the MRIs may be periodically scheduled to generally assess new disease activity (gadolinium-enhancing lesions on post-contrast T1 images) and/or evidence of MRI worsening (greater lesion volumes, reduced brain or spinal cord volume), or they may be taken to determine whether a new potential or actual symptom likely corresponds to an exacerbation or to determine whether a current treatment has been effective in slowing or halting progression of the disease.

Thus, a care provider may determine whether and/or an extent to which a subject is experiencing multiple sclerosis with ongoing worsening based on one or more of: a frequency and severity of new symptoms; a worsening of old symptoms; whether symptoms affect a previously unaffected neurologic domain or distribution; new or worsening clinical disability; development of new lesions; a change in lesion load; a change in MRI volume; and/or subject self-assessments. Any of these determinations may be identified in noisy assessment record 110.

Various scales have been developed to more objectively assess disease worsening in multiple sclerosis. Some scales are designed to assess well-being of a subject and are based on subjects' responses to a series of questions.

Some scales are based on a care provider's assessments of a subject's functional abilities. One such functionality-based scale is the Expanded Disability Status Scale. This scale captures multiple neurologic domains, but is primarily influenced by a subject's ambulatory ability. The scale ranges from 0 to 10 and is discretized by 0.5 increments. Scores between 0 and 4.5 are distinguished based on a quantity of functional systems for which a subject is experiencing a symptom or disability and a severity of the symptom/disability. Scores between 4.5 and 7.5 are distinguished based on how far a subject can move and whether and/or whether and/or which types of walking aids are needed for the movement. Scores between 7.5 and 9.5 are distinguished based on a degree to which a subject is restricted to bed and retains any function. A score of 10 is indicative of death due to multiple sclerosis. Noisy assessment record 110 may identify a progression or disability metric.

III.B. Pre-Processing

Data extraction system 105 may include one or more pre-processing controllers to pre-process at least one of the set of noisy assessment records 110. For example, the pre-processing may include cropping, sharpening and/or straightening a noisy assessment record 110. The pre-processing may additionally or alternatively include changing a magnification, color scheme, a file type, contrast, and/or dimensions of a noisy assessment record 110.

A pre-processing controller can include a character recognition controller 120, which can pre-process a noisy assessment record 110 to detect each of multiple characters (e.g., each letter, number, and/or symbol) in the noisy assessment record 110. For each character that was detected, character recognition controller 120 can further identify a location or portion of the noisy assessment record 110 that depicts the character. Relative locations of different characters may then be calculated and used to detect one or more character strings (e.g., codes, words, abbreviations, etc.) that are depicted within the noisy assessment record 110. For each noisy assessment record 110, character recognition controller 120 can store a character recognized record 125 that includes the characters and/or the character string(s) that were detected. Character recognized record 125 can further include an indication of where, within the original noisy assessment record 110, the characters and/or character string(s) that were detected were located.

In some instances, character recognized record 125 is a new version of the noisy assessment record 110 that was generated by character recognition controller 120 and that includes the characters and/or the character strings that were detected. The new version of the noisy assessment record 110 may (but need not) further include the original noisy assessment record 110. For example, the new version may include an image that is the original noisy assessment record 110 and metadata that includes the character string(s) that were detected.

III.C. Code Detector

Record decipher system 113 includes a code detector 130 that processes the characters and/or character strings that were detected to determine whether the characters and/or character strings include or correspond to any of a set of codes. A code may be (for example) a word, acronym, root word, abbreviation, or combination thereof. A code may include a selection, an image, a check, etc. that (for example) corresponds to a presented list item.

Code detector 130 may identify the set of codes based on previous input from a user and/or by retrieving the set of codes from a data store. In some instances, code detector 130 retrieves one or more root codes from a data store. Code detector 130 can define multiple codes for each root code (e.g., to represent potential differences in parts of speech, plural versus singular, misspelling, abbreviations, etc.) and may then define the set of codes to include the multiple codes corresponding to the one or more root codes. Alternatively or additionally, the set of codes may include the one or more root codes.

The set of codes can include one or more codes representing a relapse, one or more codes representing progression, and one or more codes representing a type of multiple sclerosis. A code that represents a relapse may include (for example) “relapse”, “relaps*”, “relapsing”, “new”, a code indicative of a new symptom (e.g., representing a symptom not identified in a previous noisy assessment record), a code that indicates observation of a new lesion, a code that indicates observation of an enhancing lesion, and/or a code that is indicative of a prescription of a steroid (e.g., methylprednisolone). A code that represents progression may include (for example) “progressing”, “progress*”, “worse*”, a code that indicates use of a walking aid not previously used (e.g., a cane, bilateral ambulatory assistance, a walker, or a wheelchair), and/or a code that indicates brain atrophy. A code that represents a type of multiple sclerosis may include (for example):
A code that represents a relapsing type of multiple sclerosis, such as “relapsing multiple sclerosis”, “relapse remitting multiple sclerosis”, “relapsing”, “relapse”, “RMS”, or “RRMS”,
A code that represents a secondary progressive type of multiple sclerosis, such as “secondary progressive” or “SPMS”;
A code that represents a primary progressive type of multiple sclerosis, such as “primary progressive” or “PPMS”;
A code that represents a progressive relapsing multiple sclerosis, such as “progressive relapsing” or “PRMS”; or
A code that represents clinically isolated syndrome, such as “clinically isolated”, “isolated” or “CIs”.

In some instances, code detector 130 disregards punctuation (e.g., hyphens) and/or capitalization when determining whether a noisy assessment record 110 includes an instance of a given code. Alternatively, the set of codes that may be used as a reference may include multiple character strings that differ from each other only based on punctuation and/or capitalization.

In some instances, code detector 130 uses one or more search operators to detect a term. For example, an “OR” search operator may be used to define a first search component as any of “relapse remitting”, “relapsing remitting”, “RR”, or “relapsing” and to further define a second search component as any of “multiple sclerosis”, “MS”, “type”, or “phenotype”. A proximity operator (e.g., “near/#”, “before/#”, and/or “after/#”) may be used to indicate that in order to detect a given term, the first term must appear within a specified number of words and/or within a specified number of characters. This may facilitate detecting that content that states that a subject has a “relapsing phenotype of the previously diagnosed disease of multiple sclerosis” may still be interpreted by code detector 130 as conveying an interpretation of a corresponding care provider that the subject had relapsing multiple sclerosis at the corresponding evaluation time.

In some instances, code detector 130 uses a neural network to generate some or all of the set of terms and/or to determine whether characters and/or character strings that were detected include a term in the set of terms. For example, code detector 130 may use a natural language processing technique that learns cluster definitions and/or a topology to predict whether a given word, character string, and/or set of characters corresponds to any of one or more predefined types of multiple sclerosis. Code detector 130 may then use cluster definitions and/or topology to determine to which cluster or arm a given character string corresponds (if it corresponds to any cluster or arm). Each word, character string, and/or set of characters that is determined to correspond to a cluster or arm of a topology can be defined to be a code.

Code detector 130 can store code detection information 135, which can identify each code that code detector 130 detected in a noisy assessment record 110 (corresponding to a code instance). Code detection information 135 can further identify a particular noisy assessment record 110 within which the code instance was detected and/or where within the particular noisy assessment record 110 the code instance was located.

III.D. Time Stamp Detector

Data extraction system 105 includes a time stamp detector 140 that identifies a time stamp to be associated with each code instance. The time stamp may be or may include a date, a month-year combination, a year, a quarter, etc.

The time stamp may be detected as a time that corresponds to an underlying assessment record 110 from within which the code instance was detected. In some instances, the time stamp indicates a time at which the noisy assessment record 110 was created or a time at which the noisy assessment record was saved. In some instances, the time stamp indicates a time at which initial results (e.g., indicating a laboratory value or imaging value) were collected, where the noisy assessment records 110 characterize or interpret the initial results.

Time stamp detector 140 may store the detected time stamps 145. Each time stamp may be stored in association with an identifier of: a corresponding code instance (corresponding to a subject), a corresponding subject, and/or a corresponding noisy assessment record 110.

III.E. Noisy Assessment Data

Data extraction system 105 stores codes, time stamps, and identifiers that were detected as noisy assessment data 112. Noisy assessment data 112 can include multiple data elements. In some instances, each data element corresponds to a single code instance, and the data element includes or is associated with an identifier of a given code that was detected, an identifier of a subject, a time stamp associated with the code instance, and potentially an identifier of an underlying noisy assessment record 110. In some instances, each data element corresponds to a single noisy assessment record 110, and the data element includes an identifier of a subject, a time stamp associated with the noisy assessment record 110, each code instance that was detected within the noisy assessment record 110, and potentially an identifier of the noisy assessment record 110. For example, the data element may indicate that “RMS” was detected twice within the noisy assessment record 110, “relapse” was detected once, and “progress” was detected once.

Thus, a data store with the data elements of noisy assessment data 112 can be queried to identify codes that are associated with a particular subject and with time stamps that are within a time period of interest. In some instances, data extraction system 105 controls the data store and may query the data store for noisy assessment data 112 that can then be availed to (e.g., transmitted to) record decipher system 113. In some instances, the data store is made accessible to record decipher system 113, such that record decipher system 113 can retrieve noisy assessment data 112 (e.g., corresponding to a subject and potentially to a time period) from the data store.

III.F. Type Inference Logic Implementor

Record decipher system 113 includes type inference logic implementor 150, which uses the code instances to determine an inferred type 155 of multiple sclerosis associated with a given subject (e.g., and a given time period). Type inference logic implementor 150 infers the type of multiple sclerosis based on code detection information 135 and potentially based on detected time stamps 145.

While mapping between one or more codes to a corresponding type of multiple sclerosis may be straight-forward, it may be more difficult to map other codes. For example, if noisy assessment data 112 includes “relapse”, the use of the term may indicate that the subject is experiencing a relapse (and may have either of relapsing multiple sclerosis or progressive relapsing multiple sclerosis) or that a symptom is potentially indicative of a relapse.

There are multiple reasons why mapping a code to a given type of multiple sclerosis is complicated. Different phenotypes of multiple sclerosis have overlapping clinical indicators. For example, a “relapse” may be consistent with relapsing multiple sclerosis or with progressive relapsing multiple sclerosis, and “progression” may be consistent with nearly all types of multiple sclerosis (potentially with the exception of clinically isolated syndrome). Additionally, noisy assessment data 112 or an underlying noisy assessment record 110 may indicate that a circumstance is interpreted as a possible relapse, likely progression, etc., where the care provider may be waiting for further information or data to determine whether the circumstance represented a real relapse or true progression. Further, there are many variations in each of multiple potential terms used in the context of multiple sclerosis. For example, relapsing multiple sclerosis may be represented as any of: relapsing multiple sclerosis, relapsing remitting multiple sclerosis, relapsing-remitting multiple sclerosis, relapse remitting multiple sclerosis, relapse-remitting multiple sclerosis, relapsing MS, relapsing remitting MS, relapsing-remitting MS, relapse remitting MS, relapse-remitting MS, RRMS, RR MS, RMS, RR multiple sclerosis, relapsing multiple sclerosis, or relapsing MS.

Type inference logic implementor 150 may determine inferred type 155 of multiple sclerosis based on (for example) a distribution (e.g., of multiple sclerosis types or of codes) corresponding to the code instances and/or temporal dynamic based on the code instances or based on multiple sclerosis types (identified based on the code instances). For example, each code instance may be mapped to a corresponding type of multiple sclerosis (e.g., based on a mapping of each code to a type of multiple sclerosis), and a distribution may identify a number or percentage of code instances associated with each of multiple types. Type inference logic implementor 150 may then infer that the subject was experiencing a type of multiple sclerosis associated with a highest number or highest percentage of the codes that were detected.

In some instances, type inference logic implementor 150 generates an inferred type 155 of multiple sclerosis associated with a particular time point or time period, which may be inferred based on multiple code instances. For example, noisy assessment data 112 may include code instances corresponding to multiple codes, and type inference logic implementor 150 may identify a single inferred type 155 of multiple sclerosis to associate with the time point or time period using the multiple code instances. As another example, noisy assessment data 112 may include code instances corresponding to multiple codes associated with multiple time stamps 145, and type inference logic implementor 150 may detect a trend, interpolate, or extrapolate to predict an inferred type 155 of multiple sclerosis to be associated with a given time point or time period.

Type inference logic implementor 150 may use (for example) a look-up table, one or more type criteria 160 and/or one or more machine-learning models to predict how codes map to types of multiple sclerosis. For example, each of type criteria 160 may correspond to a different type of multiple sclerosis. Thus, if a given type criterion 160 is satisfied, type inference logic implementor 150 may infer that the subject has the corresponding type of multiple sclerosis. In some instances, type criteria 160 may be configured such that only a single criterion is to be satisfied based on a given input data set (e.g., that includes one or more code instances). In some instances, type criteria 160 may be configured such that it is possible in theory that multiple type criteria 160 may be specified, but an iterative approach (of individually assessing each type criterion 160 in a specified order until a type criterion 160 is satisfied) may result in a determination that a single type criterion 160 is satisfied.

A type criterion 160 may be used to evaluate (for example) a percentage of code instances associated with a single type of multiple sclerosis, which type of multiple sclerosis was associated with code instances associated with later time stamps in a given time period (e.g., a calendar year) or whether types of multiple sclerosis associated with code instances represent a known progression of multiple sclerosis (e.g., from relapsing multiple sclerosis to secondary progressive multiple sclerosis), etc. For example, a type criterion 160 associated with the relapsing type of multiple sclerosis may be configured to be satisfied if the majority of code instances associated with a time period are associated with the relapsing type of multiple sclerosis and less than 10% of code instances associated with the time period are associated with secondary progressive multiple sclerosis. Meanwhile, a type criterion 160 associated with the primary progressive type of multiple sclerosis (which is a chronic type of multiple sclerosis that does not precede or follow another type) may be configured to be satisfied if the a percentage of code instances associated with the primary progressive type of multiple sclerosis is higher than the percentage of code instances associated with any other type of multiple sclerosis.

In some instances, one or more type criteria (or a decision tree or order of an iterative evaluation of type criteria) may be configured to assess hysteresis. For example, a type criterion may indicate that once a given inferred type 155 of multiple sclerosis has been determined, subsequent inferred types 155 are to be the same inferred type 155, potentially unless a heightened criteria is met (e.g., a higher quantity or higher percentage of mapped types of multiple sclerosis corresponds to a given type of multiple sclerosis or any other type of multiple sclerosis aside from one which was initially identified in inferred type 155).

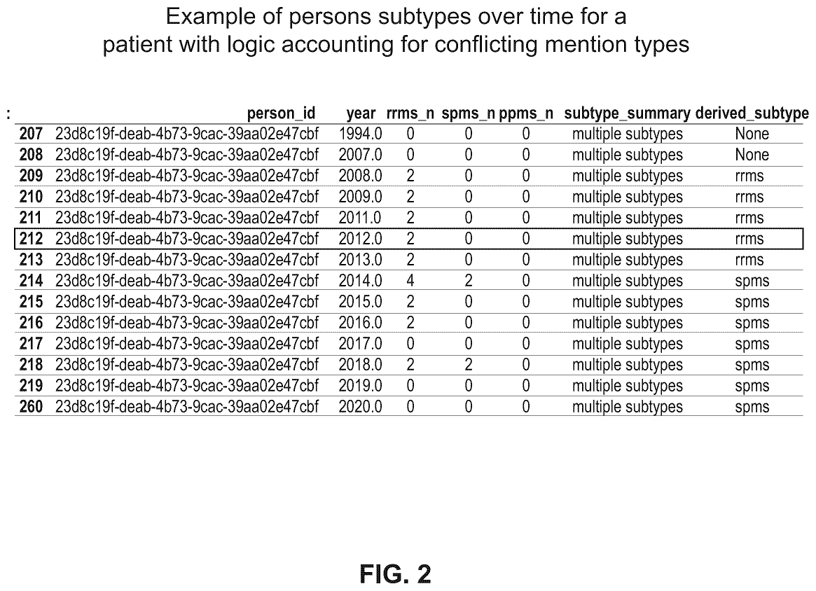

FIG. 2 shows exemplary distributions of across each of multiple types of multiple sclerosis (relapsing multiple sclerosis, secondary progressive multiple sclerosis, and primary progressive multiple sclerosis) to which code instances were mapped a given subject across 14 years. In years 1994 and 2007, there were no code instances detected in noisy assessment data 112 associated with the subject that corresponded to any of the three multiple types. In 2008, 2009, 2011, 2012, and 2013, each of the code instances was mapped to a same type of multiple sclerosis (relapsing multiple sclerosis), and the inferred type of multiple sclerosis was thus determined to be relapsing multiple sclerosis. In 2014, two of the code instances were mapped to the relapsing multiple sclerosis subtype, while four code instances were mapped to the secondary progressive multiple sclerosis. A type criterion 160 associated with secondary progressive multiple sclerosis may indicate that the criterion is satisfied when at least two code instances are mapped to secondary progressive multiple sclerosis. In 2015 and 2016, all code instances were mapped to the relapsing multiple sclerosis type. However, in terms of current scientific understanding, a progressive form of multiple sclerosis cannot transition into relapsing multiple sclerosis. Thus, a type criterion 160 may indicate secondary progressive multiple sclerosis is chronic and that once it is determined that secondary progressive multiple sclerosis is mapped to a time period for a given subject, code instances of future time periods for the subject are to remain mapped to secondary progressive multiple sclerosis.

In some instances, different type criteria 160 are used for different subjects. For example, a different set of type criteria 160 may be defined for each possible initial diagnosis of a type of multiple sclerosis. For example, type inference logic implementor 150 may identify an earliest time stamp 145 associated with a subject, may determine to which type of multiple sclerosis the code(s) associated with the earliest time stamp 145 corresponds, and may then select a set of type criteria 160 to use based on the type of multiple sclerosis.

In some instances, two or more type criteria 160 are integrated together to form a decision tree. In some instances, type inference logic implementor 150 iteratively assesses each criterion of type criteria 160. Type criteria 160 may be assessed in a predetermined order, where the order may be based on which pairs of types of multiple sclerosis may occur within a disease trajectory of a single subject, how frequently a given type of multiple sclerosis is misdiagnosed as another type of multiple sclerosis, a particular history of the subject with respect to diagnoses or inferences of types of multiple sclerosis, etc.

For example, primary progressive multiple sclerosis is a chronic type of multiple sclerosis, in that primary progressive multiple sclerosis does not progress to another type of multiple sclerosis did it arise due to progression from another type of multiple sclerosis. It may be unlikely that new evidence would indicate that a diagnosis of primary progressive multiple sclerosis was erroneous and should have instead been secondary progressive multiple sclerosis, as that would require having evidence about the subject having relapses before the disease became progressive and uncovering previously unrecognized symptomatic data from the past may be a rare occurrence. However, if the subject experiences a relapse and remission (e.g., where a symptom arises and then disappears), this new data may indicate that the subject has progressive relapsing multiple sclerosis or potentially even relapse multiple sclerosis.

Thus, the order may correspond to a probability that a subject having been initially diagnosed with primary progressive multiple sclerosis will later be diagnosed with a given (same or different) type. To illustrate, an order for evaluating type criteria 160 may indicate that type criteria 160 associated with the following types of multiples sclerosis are to be evaluated in this order: primary progressive multiple sclerosis, primary relapsing multiple sclerosis, relapsing multiple sclerosis, clinically isolated syndrome, relapsing multiple sclerosis, undetermined. Meanwhile, if the subject was initially diagnosed with clinically isolated syndrome, the order used to evaluate type criteria 160 may correspond to the following order: clinically isolated syndrome, relapsing multiple sclerosis, relapsing progressive multiple sclerosis, secondary progressive multiple sclerosis, and primary progressive multiple progressive.

For a given inferred type 155 of multiple sclerosis, type inference logic implementor 150 may generate a confidence of inferred type 155 being accurate for a corresponding subject and time period (or time point). The confidence may be based on (for example), noise of code instances, noise across types of multiple to which code instances were mapped, etc.

Type inference logic implementor 150 may store each inferred type 155 in a local or remote data store. Each inferred type 155 may be stored in association with an identifier of a subject, time stamp, time period, and/or confidence.

Type inference logic implementor 150 may generate a timeline that includes an ordered set of inferred types 155 of multiple sclerosis. Information presented with the inferred types 155 or a relative spacing between representations of each inferred type 155 may represent an estimate as to how diagnoses of the subject's multiple sclerosis changed over time and/or how the type of the subject's multiple sclerosis changed over time.

III.G. Modulation Determination Logic

A record decipher system 113 includes modulation determination logic implementor 165, which uses modulation determination logic to determine a modulation 170 associated with the code instances (represented in code detection information 135). Modulation 170 may correspond to a time period associated with data underlying the generation of a single inferred type 155 or multiple inferred types 155. Modulation 170 may include a numeric value, a categorical value, or a binary value.

Modulation 170 may characterize a distribution of the code instances or a temporal dynamic based on the code instances. For example, modulation determination logic implementer 165 may map each code instance to a multiple sclerosis type. The mapping may be performed using (for example) a look-up table, one or more predefined rules, and/or results generated by a machine-learning model (e.g., identifying cluster definitions).

Modulation 170 may indicate whether and/or an extent to which inferred type 155 of multiple sclerosis varies over a time period and/or is changing over a time period. Modulation 170 may indicate or relate to an extent to which inferred type 155 of multiple sclerosis is reliable and/or a probability that a subject is experiencing ongoing worsening.

In some instances, modulation determination logic implementor 165 maps each code (and thus each code instance) to a corresponding type of multiple sclerosis. The mapping may be performed (for example) using a look-up table or machine-learning model. Modulation determination logic implementor 165 may detect a shift or change in the corresponding types of multiple sclerosis across the time window. Modulation determination logic implementor 165 may then detect a shift across types of multiple sclerosis during the time period.

Modulation determination logic implementor 165 can store one or more modulations 170. Modulation 170 may indicate whether or an extent to which an inferred type 155 of multiple sclerosis varies or shifts across a time period. For example, modulation determination logic implementor 165 may identify a percentage of code instances associated with each of type of multiple sclerosis, and modulation 170 may identify the highest percentage and may further indicate the corresponding type of multiple sclerosis.

Modulation 170 further or alternatively represent a confidence of assigning a given type of multiple sclerosis to a time period. A low confidence may indicate a relatively high degree of variability across code instances and/or types of multiple sclerosis to which code instances were mapped.

III.H. Alert Generation Logic Implementor

Record decipher system 113 includes alert generation logic implementor 175, which uses modulation 170 and alert generation logic to determine whether to generate an alert. The alert generation logic can be configured to determine whether any of one or more alert criteria 180 is satisfied. Alert criteria 180 can include an inconsistency alert criterion that is configured to be satisfied when modulation 170 represents a relatively high degree of noise across code instances and/or types of multiple sclerosis to which code instances were mapped. For example, the high-noise alert criterion may be configured to be satisfied if modulation 170 (e.g., a numeric value) is above a threshold, if modulation 170 (e.g., a categorical value) corresponds to a particular category or any of a set of categories, or if modulation 170 (e.g., a binary value) corresponds to a particular binary value. As another example, the high-noise alert criterion may be configured to be satisfied if modulation 170 includes a confidence metric, and the confidence metric is below a threshold.

Alert criteria 180 can include a transition alert criterion that is configured to be satisfied when modulation 170 represents that a shift has been detected between types of multiple sclerosis. Alert criteria 180 may alternatively or additionally include a progression alert criteria that is configured to be satisfied when modulation 170 represents that a shift has been detected that represents progression of types of multiple sclerosis (e.g., from relapsing multiple sclerosis to secondary progressive multiple sclerosis). Alert criteria 180 may alternatively or additionally include a diagnosis correction alert criteria that is configured to be satisfied when modulation 170 represents that a shift has been detected between two types of multiple sclerosis that do not occur in a disease path. For example, it is not possible for a single subject to have had both “primary progressive multiple sclerosis” and “relapsing multiple sclerosis”. Thus, if code instances corresponding to a single subject are mapped to each of these types of multiple sclerosis, this may represent that a care provider is changing or correcting a previous diagnosis. Alert criteria 180 may alternatively or additionally include a stability alert criterion that is configured to be satisfied when modulation 170 is below a threshold a represents a stability in a type of multiple sclerosis and/or a lack of progression. For example, the stability alert criterion may be configured to be satisfied if it is inferred that the subject's type of multiple sclerosis has remained as the relapsing type for at least a predefined number of years.

III.1. Alert Generator

Record decipher system 113 includes an alert generator 185 that generates an alert 190 when alert generation logic implementor 175 has determined that an alert criterion 180 is satisfied. Alert 190 may include text that identifies what the alert represents (e.g., noise, a shift in multiple sclerosis types, progression, a diagnosis correction, etc.).

The alert may include code detection information 135 (e.g., one or more codes corresponding to a time period and/or one or more code instances corresponding to a time period) and/or modulation 170. Alert 190 may indicate how each of one or more code instances were mapped to a type of multiple sclerosis. Alert 190 may identify an inferred type 155 of multiple sclerosis determined for a given time period. Alert 190 may include one or more links that enable a viewer to access one or more underlying noisy assessment records 110.

III. J. Output Interface

Record decipher system 113 includes output interface 195 that outputs inferred type 155 of multiple sclerosis. When alert generation logic implementor 175 determines that an alert is to be generated, output interface 195 may further (e.g., concurrently or separately) output the alert. Alert generation logic implementor 175 may further output an identifier of the subject and/or other information about the subject (e.g., age, sex, co-morbidities, etc.).

Output interface 195 may include (for example) a software component that generates a webpage or message (e.g., an email) and a transmitter to transmit the webpage message. The webpage or message may include inferred type 155 and any alert. The webpage or message may include a timeline generated by type inference logic implementor 150 that indicates relative timing of inferred types 155 of multiple sclerosis for an individual subject.

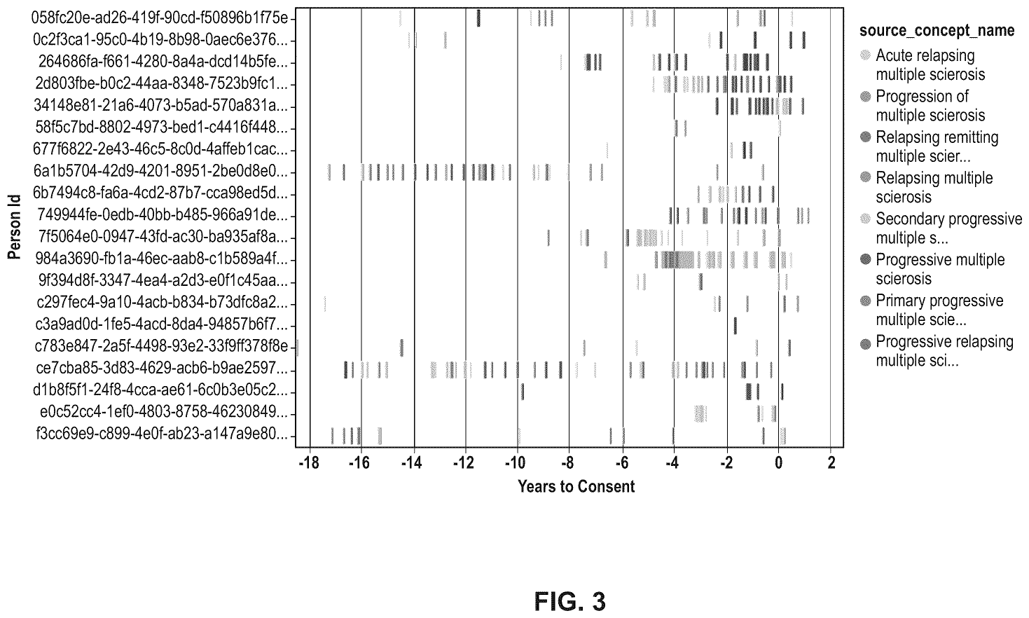

The webpage or message may include a code instance event chain (e.g., generated by code detector 130) that shows relative timings of different code instances detected in noisy assessment data 112 associated with a given subject. FIG. 3 shows exemplary code event chains for twenty subjects (where each row corresponds to a subject). Each tick corresponds to a code instance. The color of the tick indicates which code instance was detected. The horizontal position of the tick indicates how the time stamp 145 associated with the code instance compared to other time stamps associated with the same subject. The code event chain demonstrates the noise that may be present across code instances.

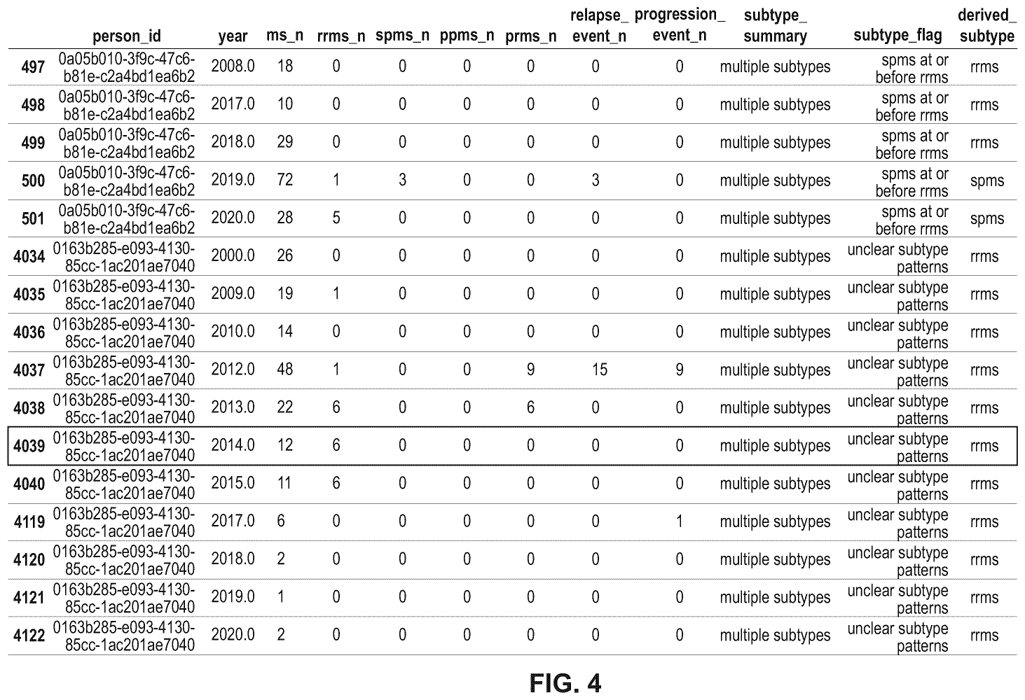

The webpage or message may identify one or more inferred types 155 of multiple sclerosis (e.g., in addition to identifying one or more code instances). FIG. 4 shows an exemplary interface that identifies code instances and inferred types 155 of multiple sclerosis for two subjects. Each line corresponds to a given year (identified in the third column). The first five lines correspond to a first subject, and the remaining eleven lines correspond to a second subject. The values in the fourth through tenth columns indicate how many times a code (identified in the label for the column) was detected in noisy assessment data 112 associated with the year and subject. For example, the code “ms” or “multiple sclerosis” was detected 18 times across noisy assessment data 112 corresponding to the first subject and year 2008. The values in the last column identify the inferred type 155 of multiple sclerosis for the year and subject. For example, the inferred types 155 indicate that it was predicted that the first subject had relapsing multiple sclerosis between 2008 and 2019 and secondary progressive multiple sclerosis in 2019-2020 and that the second subject had relapsing multiple sclerosis between 2000 and 2020.

The values in the second to last column identify any alert that was generated for the subject and year. For example, an alert was generated for the first subject because at least one code instance corresponding to the secondary progressive multiple sclerosis was detected in a same or previous year as one or more code instances corresponding to the relapsing multiple sclerosis code. (Specifically, five instances of relapsing multiple sclerosis were detected in 2020, though three instances of secondary progressive multiple sclerosis were detected in 2019.) A transition from the secondary progressive type to the relapsing type of multiple sclerosis is not possible, so it may be advantageous for a care provider to review noisy assessment records 110 to determine whether any correction in the records and/or of a diagnosis is warranted.

As another example, an alert was generated for the second subject because code instances corresponding to each of the relapsing multiple sclerosis code and the progressive relapsing multiple sclerosis code were detected. It is not possible for a same subject to have experienced both of these types of multiple sclerosis, so it may be advantageous for a care provider to review noisy assessment records 110 to determine whether any correction in the records and/or of a diagnosis is warranted.

The webpage can include one or more input options configured to receive input that (for example) confirms or overrides an inferred type; confirms or overrides a code instance; confirms or overrides a mapping of a code (or code instance) to a type of multiple sclerosis; acknowledges an alert; confirms or refutes that noisy assessment data 112 indicate that the subject has progressed over a time period; and/or confirms or refutes that noisy assessment data 112 indicate that a correction of a diagnosis of a type of multiple sclerosis has occurred.

In some instances, output interface 195 may include a test-order input component configured to receive input to order a medical test (e.g., an MRI), a consultation input component configured to receive input to request a consultation with another care provider, and/or a reassessment input component configured to receive input to request an appointment with the subject. The test-order input component, consultation input component, and/or reassessment input component may be presented when (for example) an alert identifies noise across mapping of codes and/or when an inferred type 155 of multiple sclerosis was unknown. It will be appreciated that a user may alternatively order a test, request a consultation, and/or request a reassessment using another interface or system.

III.K. Advantages

Record decipher system 113 provides technical advantages of converting noisy data into an actionable and interpretable code that can be used to influence a diagnosis, treatment, clinical-study enrollment or retrospective assessment of a subject. The Record decipher system 113 can be an automated and scalable system that can process large amounts of noisy assessment data 112. Further, type inference logic implementor 150, modulation determination logic implementor 165, and/or alert generation logic implementor 175 may be configured to dynamically adapt to new types of data and/or data patterns by updating (for example) type criteria 165, modulation definitions, and/or alert criteria 180. Further, alert generation and presentation facilitate efficient and focused review of noisy assessment data. This review can improve the likelihood that a care provider recognizes potentially important transitions between types of multiple sclerosis and/or spends additional attention to resolve inconsistent and/or unphysiological patterns.

### IV. Exemplary Process for Processing Noisy Assessment Records to Generate Inferred Types of Multiple Sclerosis and Alerts

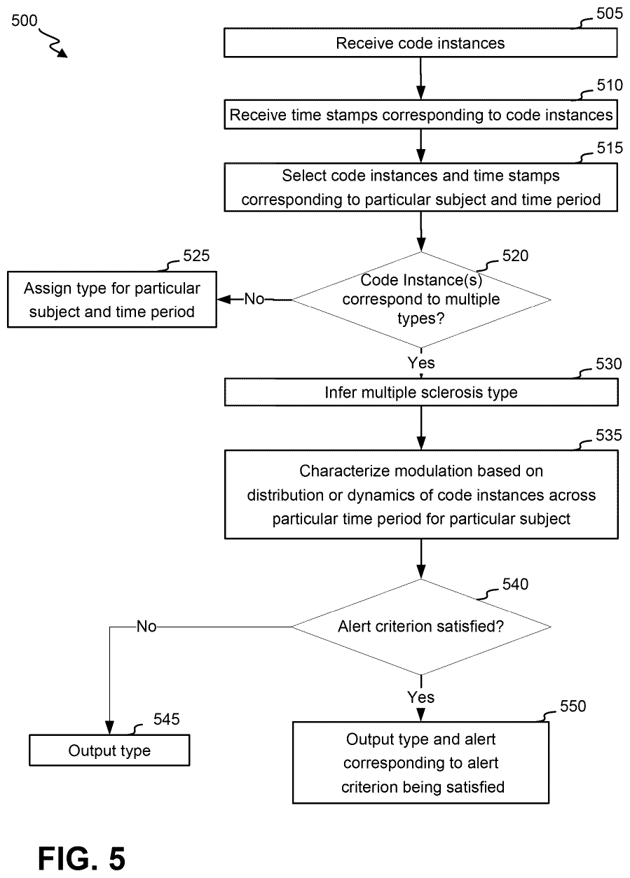

FIG. 5 shows a flowchart for a process 500 of inferring multiple sclerosis types and/or conditionally outputting alerts based on noisy assessment data. Process 500 begins at block 505 where record decipher system 113 receives a set of code instances. For example, record decipher system 113 may receive the set of code instances (e.g., and/or any additional corresponding code detection information 135) from data extraction system 105 and/or from noisy assessment data 112. The set of code instances may have been detected by code detector 130 from within one or more noisy assessment records 110. Each of the set of code instances may identify a code that was detected and/or a root code that corresponds to the detected code.

At block 510, record decipher system 113 receives a set of time stamps that corresponds to the set of code instances. The set of time stamps may be received from (for example) data extraction system 105 and/or noisy assessment data 112. The time stamps may have been detected by time stamp detector 140 from within one or more noisy assessment records 110 and/or from metadata associated with noisy assessment records 110.

At block 515, record decipher system 113 selects a subset (or all) of the received code instances and of the time stamps that correspond to a particular subject and a particular time. In some instances, each of the code instances received at block 505 and each of the time stamps received at block 510 correspond to a particular subject (e.g., identified in a request from record decipher system 113). In some instances, each of the code instances received at block 505 and each of the time stamps received at block 510 correspond to a particular time period (e.g., identified in a request from record decipher system 113). For example, a request from record decipher system 113 may identify the particular subject and the time period (or a larger time period that includes the time period), and the code instances and time stamps may be received in response to the request. In some instances, the code instances received at block 505 and/or the time stamps received at block 510 may correspond to multiple subjects and/or a time interval that extends beyond the time period (in one or more directions), and block 515 includes filtering the code instances and/or time stamps.

At block 520, type inference logic implementor 150 determines whether the selected code instances correspond to multiple types of multiple sclerosis. Type inference logic implementor 150 may map each of the code instances to a given type of multiple sclerosis (e.g., using a technique or strategy disclosed in Section III.F). Type inference logic implementor 150 may determine whether (in various instances) all, at least a threshold quantity, or at least a threshold percentage of the selected code instances correspond to a single type of multiple sclerosis.

If so, process 500 proceeds to block 525, where the single type of multiple sclerosis is assigned in association with the particular subject and particular time period. In some instances, output interface 195 may output an indication that the single type of multiple sclerosis has been assigned in association with the particular subject and particular time period.

If type inference logic implementor 150 determines that (in various instances) all, at least a threshold quantity, or at least a threshold percentage of the selected code instances do not correspond to a single type of multiple sclerosis, process 500 proceeds to block 530, where type inference logic implementor 150 infers a given type of multiple sclerosis for the particular subject for the time period. The given type of multiple sclerosis may be inferred (for example) using a technique or strategy disclosed in Section III.F and/or by evaluating one or more type criteria 160. The given type of multiple sclerosis may be inferred based on (for example) a distribution based on the code instances and/or a temporal dynamic based on the code instances or a distribution based on the code instances.

At block 535, a modulation can be identified using a technique (for example) disclosed in Section III.G. The modulation can characterize a temporal dynamic based on the set of code instances or a distribution based on the set of code instances Each of the set of code instances used to characterize or identify the modulation can be associated with a time stamp that is within the defined time period.

At block 540, alert generation logic implementor 175 can determine whether an alert criterion (e.g., any of alert criteria 180) is satisfied using the modulation. Alert generation logic implementor 175 can determine whether the alert criterion or any alert criterion is satisfied based on an evaluation disclosed in (for example) Section III.G and/or by evaluating an alert criterion disclosed in Section III.G. For example, an alert criterion may be configured to be satisfied if it is determined that one or more metrics corresponding to code instances (or corresponding types of multiple sclerosis) are inconsistent with each other, are noisy, or are indicative of a subject potentially transitioning between different types of multiple sclerosis.

If it is determined that the alert criterion is not satisfied, process 500 proceeds to block 545, where output interface 195 outputs the type of multiple sclerosis for which it was inferred at block 530 that the particular subject had at the particular time period. If it is determined that the alert criterion is satisfied, process 500 proceeds to block 550, where output interface 195 outputs the type of multiple sclerosis for which it was inferred at block 530 that the particular subject had at the particular time period in addition to an alert corresponding to the alert criterion being satisfied. For example, the alert may correspond to one disclosed in Section III.I. or Section III. J. The alert may represent logic behind an alert criterion that was satisfied (e.g., indicating that code instances or mappings thereof were noisy and/or indicating that a trend of code instances or mappings thereof was detected).

### V. Examples

A set of codes was defined to include those listed in the left column of Table 1. The right column of Table 1 shows to which type of multiple sclerosis each of the codes was mapped.

TABLE 1

| Code | Corresponding MS Type |
|---|---|
| Acute relapsing multiple sclerosis | RMS |
| Relapsing multiple sclerosis | RMS |
| Secondary progressive multiple sclerosis | SPMS |
| Progressive multiple sclerosis | PMS |
| Primary progressive multiple sclerosis | PPMS |
| Progressive relapsing multiple sclerosis | PPMS |
| Progression of multiple sclerosis | MS with ongoing worsening |

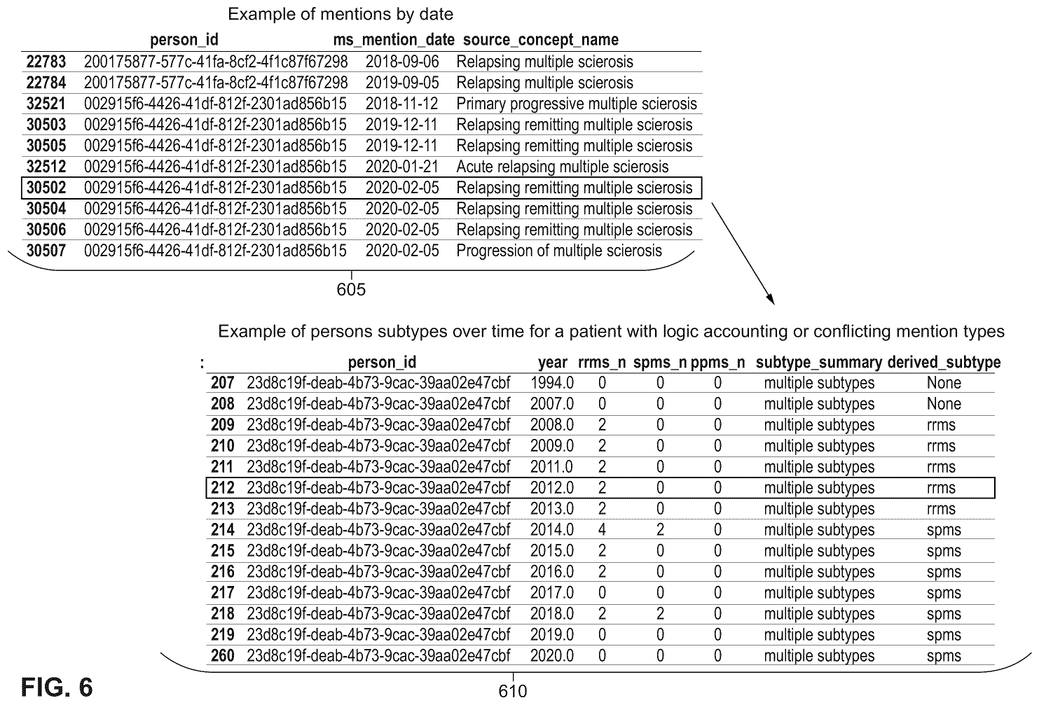

Though a single code is shown in the left column of Table 1, the code also represents any corresponding acronym, abbreviation, or variation on the term that includes a different word tense or punctuation. Noisy assessment data was accessed that corresponded to noisy assessment records (e.g., clinic notes) from the Flywheel MS underlying database. FIG. 6 shows representations of the noisy assessment data and a processed version thereof. The noisy assessment data was defined to include individual raw data elements 605, where each raw data element 605 corresponded to a detection of a code from the set of codes within an underlying noisy assessment record. Specifically, each raw data element 605 included an identifier of a subject, a time stamp associated with the underlying noisy assessment record and the code.

The noisy assessment data was processed to aggregate all entries that corresponded to a given year and to a given subject. Then, using the mappings of individual codes to types of multiple sclerosis, a distribution of representations 610 across types of multiple sclerosis was generated for each year and for each subject.

With respect to data corresponding to the underlying FlywheeIMS database, 3344 subjects were associated with at least one detection of “multiple sclerosis”, a type of multiple sclerosis, “relapse”, or “progression” in the corresponding noisy assessment records. For each of 3344 subjects, at least one of these terms was detected.

Alert criteria were defined to determine whether to flag a given inferred code or subject for an alert. The alert criteria definitions were informed by clinical expertise to note clinically implausible temporal patterns of types of multiple sclerosis.

As illustrated in Table 2, for most subjects (87%), the codes that were detected consistently mapped to a single type of multiple sclerosis. Most of these types were relapsing multiple sclerosis. Multiple types of multiple sclerosis were detected for 13% of the subjects.

TABLE 2

| subtype_summary | # Subjects | % |
|---|---|---|
| multiple subtypes | 269 | 8.0 |
| no subtype information | 1312 | 39.2 |
| Only PPMS | 119 | 3.6 |
| Only RRMS | 1532 | 45.8 |
| Only SPMS | 112 | 3.3 |

Table 3 presents similar data to that shown in Table 2, though more specific information is provided about the types of instances detected that corresponded to multiple subtypes. Some of these circumstances are consistent with medical understandings of multiple sclerosis, while others are not. For example, secondary progressive multiple sclerosis cannot precede relapsing multiple sclerosis, for 59 times subjects, this pattern of codes was detected. As another example of medical inconsistency, for 65 subjects, at least one code representing primary progressive multiple sclerosis was detected and two or more codes representing a relapse were detected.

TABLE 3

| Mappings | # Subjects | % |
|---|---|---|
| rrms only | 1532 | 75.4 |
| spms only | 112 | 5.5 |
| ppms only | 83 | 4.1 |
| unclear subtype patterns | 70 | 3.4 |
| rrms before spms | 65 | 3.2 |
| spms at or before rrms | 59 | 2.9 |
| ppms with mentions of relapse | 36 | 1.8 |
| ppms with mentions of relapse | 29 | 1.4 |
| ppms at or before rrms | 17 | 0.8 |
| rrms before ppms | 15 | 0.7 |
| majority rrms | 12 | 0.6 |
| majority ppms | 1 | 0.0 |
| majority spms | 1 | 0.0 |

For each subject and each time period, the code instances were analyzed to infer which type of multiple sclerosis that the subject had during the time period. Code instances are analyzed both by year and across all time. This is because code instances from one year may be inconsistent with (or alternatively informative of) a type that is to be inferred for another year.

Initially, code instances were evaluated per year. When code instances corresponding to a single subject and time period were mapped to different types of multiple sclerosis, the type with which the highest quantity of codes were mapped was inferred to be the type of multiple sclerosis associated with the subject and time period. When all code instances corresponding to a single subject and time period were mapped to a single type of multiple sclerosis, it was inferred that the subject had that type of multiple sclerosis. The inferred types were then reevaluated based on the code instances detected across years and/or inferred types associated with other years. For example, if only the progressive multiple sclerosis code was detected in noisy assessment data corresponding to 2015, it may be:
Inferred that the subject had primary progressive multiple sclerosis in 2015 if the primary progressive multiple sclerosis code was detected in association with another year (with no conflicting code);
Inferred that the subject had secondary progressive multiple sclerosis in 2015 if the secondary progressive multiple sclerosis code was detected in association with another year (with no conflicting code); and
Determined that the type of multiple sclerosis in 2015 was unclear if no primary or secondary progressive multiple sclerosis was detected in another year.

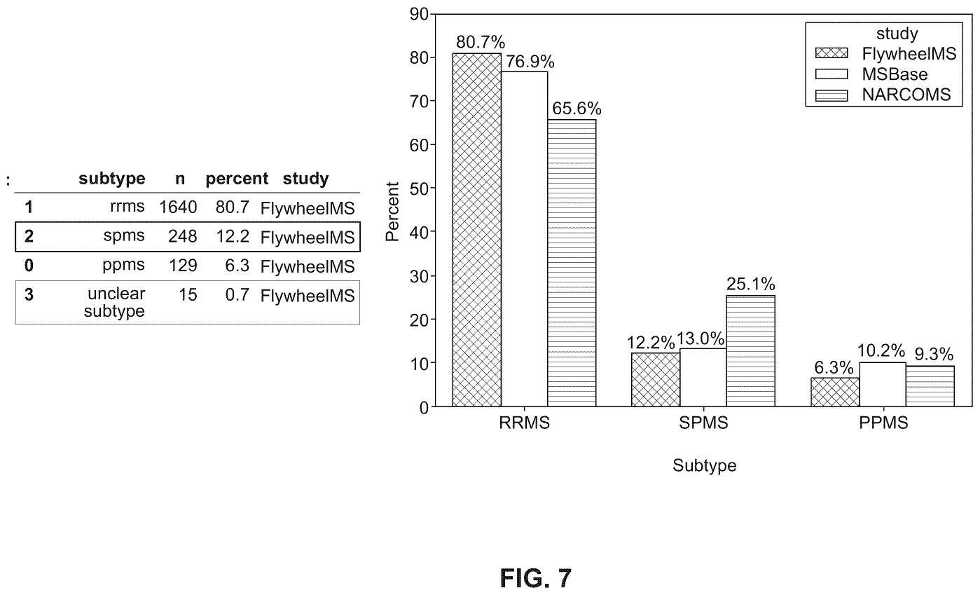

FIG. 7 shows the distribution of inferred types of multiple sclerosis across subjects (checkered bars). Further, FIG. 7 shows the distribution across types for two other populations of subjects with multiple sclerosis based on care-provider diagnoses identified in two other databases (MSBase and NARCOMS). Across all three distributions, most subjects were inferred to have or were diagnosed with having the relapsing type of multiple sclerosis. Further, for each of the three definitions, the percentage of subjects inferred or diagnosed with having secondary progressive multiple sclerosis was higher than the percentage of subjects inferred or diagnosed with having primary progressive multiple sclerosis.

For each subject, an initial subtype code was identified as a first code detected in the subject's noisy assessment data that corresponded to a subtype. In some instances, the first code was sufficient to infer that the subject had a particular type of multiple sclerosis, but in many instances (n=1525), it was not. For each first code detection, it was determined how old the subject was when the assessment was performed that corresponds to the underlying noisy assessment data.

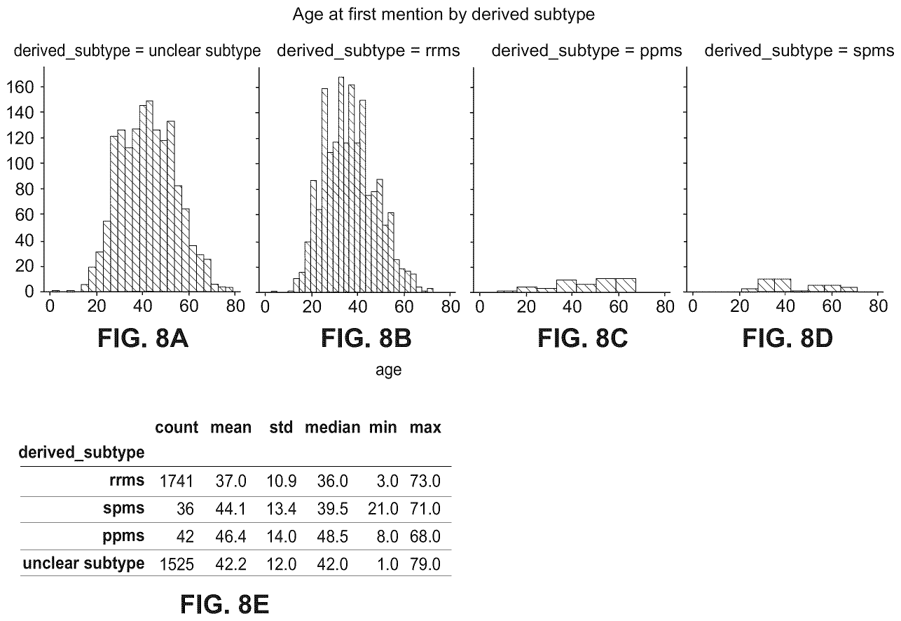

Each of FIGS. 8A-8D shows a distribution of ages of the subjects at the time when the first codes were detected. FIGS. 8A, 8B, 8C, and 8D correspond to instances where it was inferred that the subject had a given type of multiple sclerosis (unclear, relapsing, primary progressive, and second progressive, respectively) based on the first code. FIG. 8E shows statistics about the ages of the subjects corresponding to times associated with the first codes. The mean and median ages of subjects for which it was inferred that they had secondary progressive multiple sclerosis were higher than the mean and median ages of subjects for which it was inferred that they had relapsing multiple sclerosis. This is consistent with the medical understanding of how relapsing multiple sclerosis can progress to become secondary progressive multiple sclerosis (but a reverse transition is not possible). A subject may have had secondary progressive multiple sclerosis even at a first consultation because (for example) the subject recounted a past symptom history that was consistent with relapsing multiple sclerosis or because prior medical records for the subject were not available.

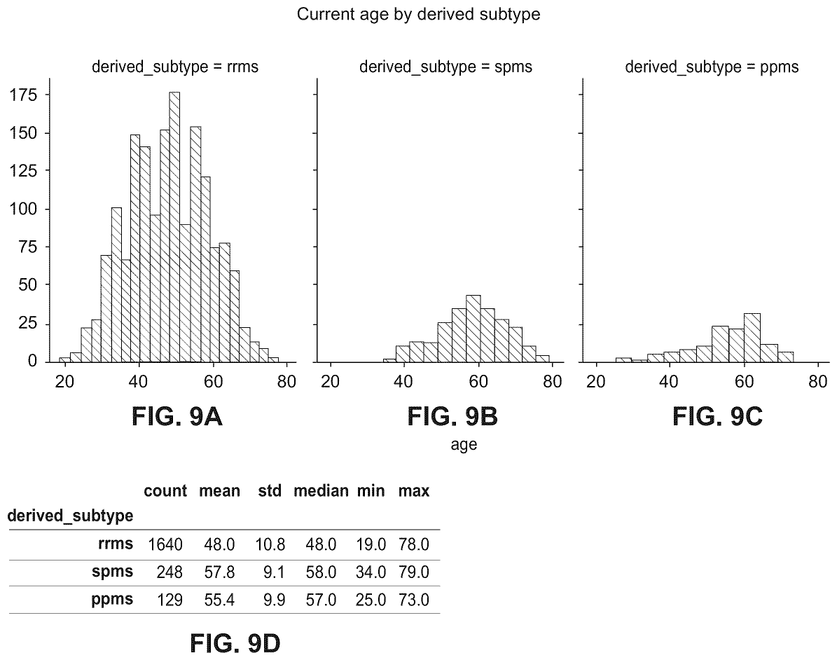

Each of FIGS. 9A-9C shows a distribution of ages of the subjects at a most recent time, and FIG. 9D shows statistics of the distribution. More subjects are represented in the two progressive multiple sclerosis types. This change is consistent with the fact that relapsing multiple sclerosis can progress to secondary progressive multiple sclerosis, and the subject set was fixed to that from an initial enrollment.

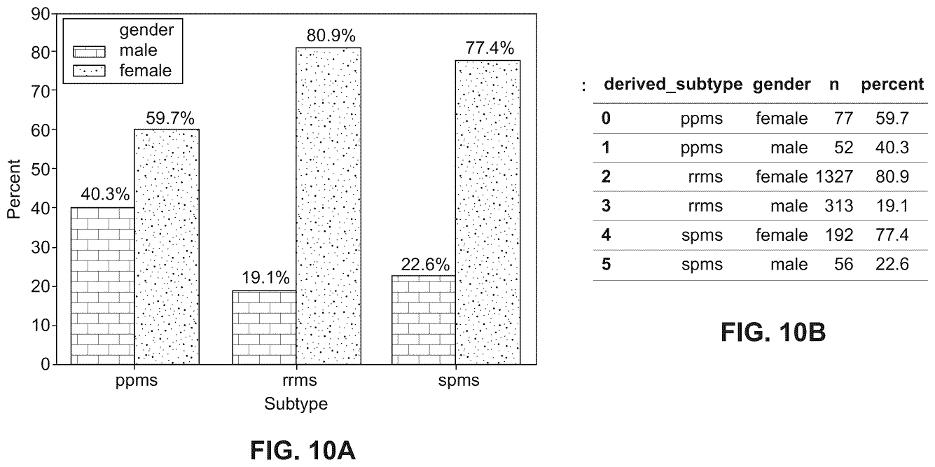

It is known that, in general, women are more likely to be diagnosed with multiple sclerosis relative to men. However, it is also known that the probability that a subject's multiple sclerosis is of the progressive primary type is higher for men than for women. Thus, the inferred types were assessed to determine whether a similar discrepancy was observed. For each of three types of multiple sclerosis (primary progressive multiple sclerosis, relapsing multiple sclerosis, and secondary progressive multiple sclerosis), a subtype population was defined to include subjects who were inferred to have the type of multiple sclerosis were identified. For each of these subtype populations, FIGS. 10A and 10B show the percentage of the subjects who were male versus female. Consistent with knowledge that more women have multiple sclerosis than men, the percentage of subjects who were women was higher than the percentage of subjects who were men across all types of multiple sclerosis. Consistent with knowledge that a man with multiple sclerosis is more likely to have the primary progressive type as compared to a woman with multiple sclerosis, 12% of the male subjects were inferred to have the primary progressive type, while only 5% of the female subjects were inferred to have the primary progressive type.

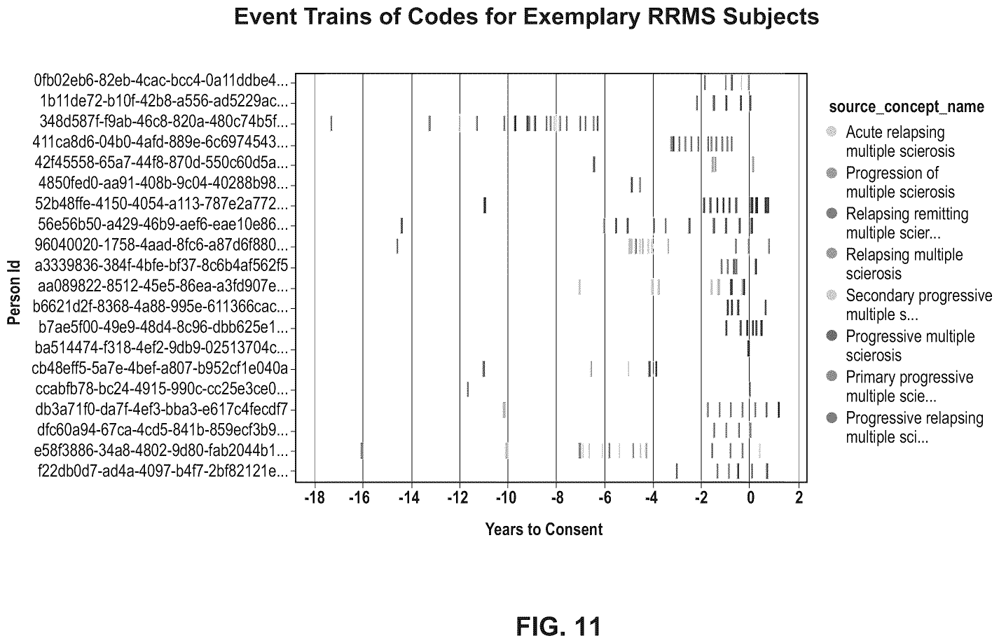

Each of FIGS. 11-15 show exemplary code event chains for twenty subjects (where each row corresponds to a subject). Each tick corresponds to a code instance. The color of the tick indicates which code instance was detected. The horizontal position of the tick indicates how the time stamp associated with the code instance compared to other time stamps associated with the same subject.

Each subject represented in FIG. 11 was inferred to have had relapsing multiple sclerosis at the year of first mention of relapsing multiple sclerosis. While a code of “progression” was detected for a few subjects, codes more specifically identifying a progressive type of multiple sclerosis (e.g., “progressive multiple sclerosis”, “primary progressive multiple sclerosis”, “progressive relapsing multiple sclerosis”, or “secondary progressive multiple sclerosis) were more commonly associated with the code instances.

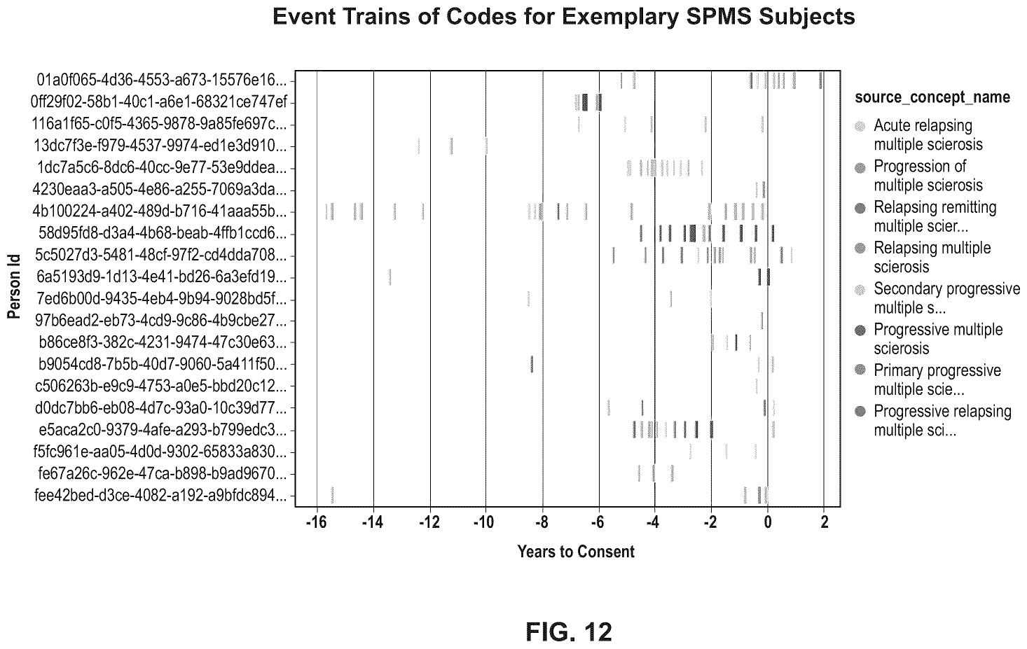

Each subject represented in FIG. 12 was inferred to have had secondary progressive multiple sclerosis at a time point associated with the year of first mention of secondary progressive multiple sclerosis. For the majority of subjects, code instances included at least one “secondary progressive multiple sclerosis” code. For other subjects, the code instances corresponded to one or more codes representing a relapsing type of multiple sclerosis and one or more codes representing progressive type of multiple sclerosis.

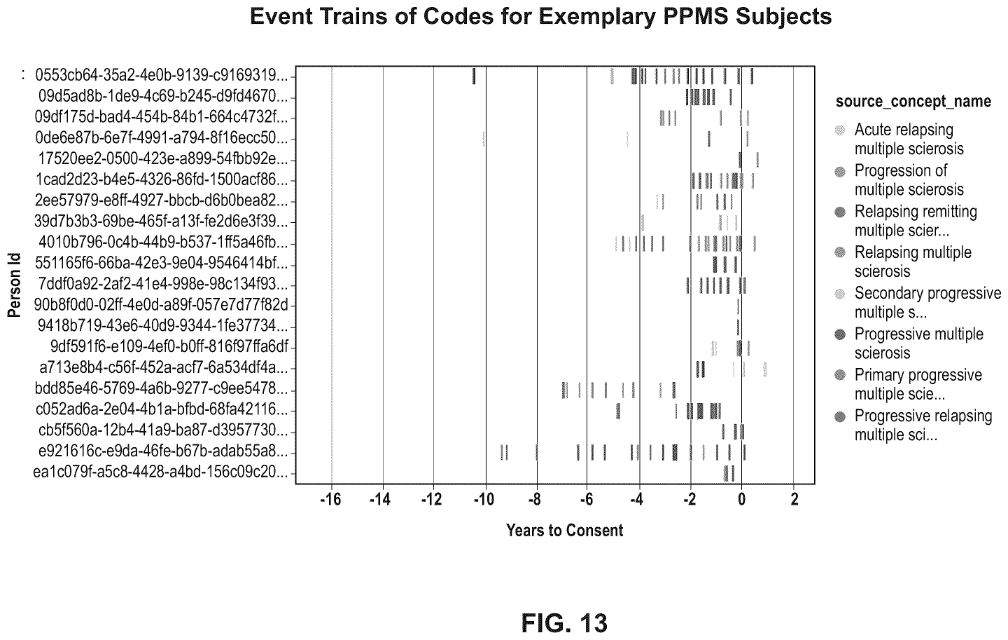

Each subject represented in FIG. 13 was inferred to have had primary progressive multiple sclerosis at a time point associated with the date of year of first mention of primary progressive multiple sclerosis. For most subjects, the vast majority or all of the code instances reference progressive or progression.

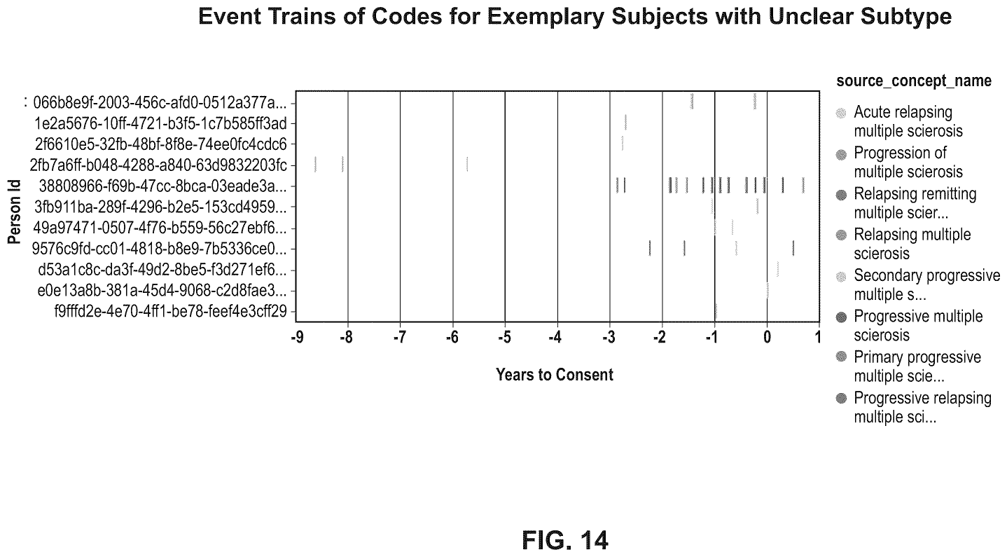

For each subject represented in FIG. 14, an inferred type of multiple sclerosis was not identified based on the codes. While instances corresponding to “acute relapsing” multiple sclerosis or “relapsing” multiple sclerosis were detected, those were considered to be insufficient to identify a specific type of multiple sclerosis according to current definitions of types of multiple sclerosis. Even though multiple code instances of relapses were detected for some subjects, the inferred type remained unclear because relapses can still occur in secondary progressive multiple sclerosis (in addition to in relapsing multiple sclerosis).

Should the definitions of the types of multiple sclerosis change (e.g., to condense the types of multiple sclerosis to be “multiple sclerosis with ongoing worsening” and “multiple sclerosis without ongoing worsening”, the code instances represented in FIG. 14 may potentially be sufficient to infer a type of multiple sclerosis. Similarly, detection of new code instances may facilitate inferring a type of multiple sclerosis even across the time periods represented in FIG. 14 (e.g., when the new code instances correspond to a single chronic type of multiple sclerosis that is consistent with the code instances represented in FIG. 14).

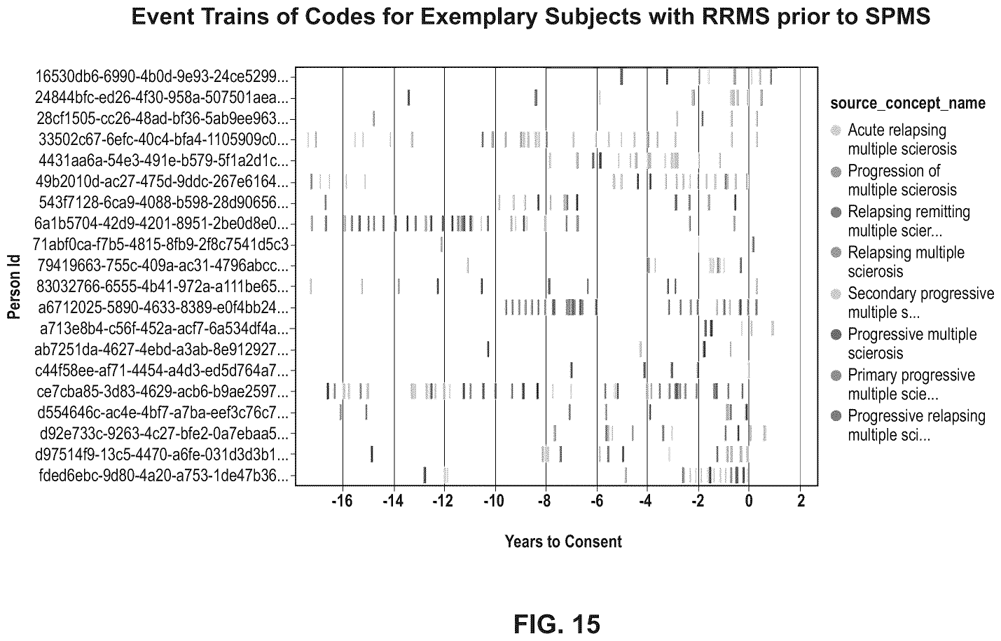

Each subject represented in FIG. 15 was inferred to have had first had relapsing multiple sclerosis across a time period associated with one or more noisy assessment data elements and to have then had secondary progressive multiple sclerosis during a subsequent time period. Thus, code instances associated with “relapsing” are associated with in early years (e.g., years before consent), whereas code instances associated with progressive subtypes and/or progression codes are associated are associated with later years.

The exemplary event chains of FIGS. 11-15 demonstrate the noise across code instances. Further, the exemplary event chains of FIGS. 11-15 demonstrate that inferred types of multiple sclerosis do correspond to real-life patterns of code instances that (through the noise) represent physiological patterns of the disease.

### VI. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A computing system comprising:
type inference logic to:
select a set of code instances within a defined time period, wherein each of the set of code instances indicates that a code of a set of codes was detected within noisy assessment data corresponding to a subject, wherein the noisy assessment data includes content originating from a care provider that identifies a characteristic of the subject or of a treatment for the subject, and wherein the subject has been diagnosed with multiple sclerosis; and
infer a type of multiple sclerosis that corresponds to the subject and a defined time period based on the set of code instances;
modulation determination logic to:
determine a temporal dynamic or distribution based on the code instances detected within the noisy assessment data; and
determine a modulation based on the temporal dynamic or distribution;
alert generation logic to determine whether an alert criterion is satisfied based on whether the modulation is above a threshold so as to represent noise or a predicted transition across types of multiple sclerosis;
an alert generator to generate an alert when the alert generation logic determines that the alert criterion is satisfied; and
an output interface to output:
the inferred type of multiple sclerosis; and
the alert when the alert generation logic determines that the alert criterion is determined to be satisfied.

2. The computing system of claim 1, wherein the type inference logic further, in response to inferring that the type of multiple sclerosis corresponds to the subject and the defined time period:
infers that the type of multiple sclerosis corresponds to the subject and another later time period that is subsequent to the defined time period.

3. The computing system of claim 1, wherein inferring that the type of multiple sclerosis corresponds to the subject and the defined time period includes:
detecting that the subject was inferred to have had another type of multiple sclerosis during another time period that was before the defined time period;
retrieving a type criterion associated with a previous inference of the other type of multiple sclerosis;
determining, based on the set of code instances, that the type criterion is not satisfied;
in response to determining that the type criterion is not satisfied, evaluating another type criterion using the set of code instances;
determining that the other type criterion is satisfied; and
determining that the type of multiple sclerosis is associated with the other type criterion.

4. The computing system of claim 1, wherein inferring that the type of multiple sclerosis corresponds to the subject and the defined time period includes:
detecting that the subject was inferred to have had another type of multiple sclerosis during another time period that was before the defined time period;
retrieving a type criterion associated with a previous inference of the other type of multiple sclerosis;
determining, based on the set of code instances, that the type criterion is not satisfied;
in response to determining that the type criterion is not satisfied, iteratively evaluating each of multiple other type criteria until one of the multiple other type criteria is satisfied; and
determining that the type of multiple sclerosis is associated with the one of the multiple other type criteria that is satisfied.

5. The computing system of claim 1, wherein the alert criterion is configured to be satisfied when the modulation indicates that the subject transitioned from a particular type of multiple sclerosis to a different particular type of multiple sclerosis, and wherein the transition is inconsistent with a definition of the different particular type of multiple sclerosis.

6. The computing system of claim 1, wherein the alert criterion is configured to be satisfied when the set code instances include a particular code instance representing a particular type of multiple sclerosis and a term representing an observation that is inconsistent with the particular type of multiple sclerosis.

7. The computing system of claim 1, wherein the inferred type of multiple sclerosis is one of: relapsing multiple sclerosis, secondary progressive multiple sclerosis, or primary progressive multiple sclerosis.

8. The computing system of claim 1, wherein the set of codes includes one or more codes representing a relapse, one or more codes representing progression, and one or more codes representing a type of multiple sclerosis.

9. The computing system of claim 1, wherein the alert criterion is configured such that a dissatisfaction of the alert criterion represents predicted disease stability.

10. The computing system of claim 1, wherein selecting the set of code instances within the defined time period includes detecting that, for each code instance in the set of code instances, a time stamp associated with the code instance is within the defined time period.

11. A computer-implemented method comprising:
selecting a set of code instances within a defined time period, wherein each of the set of code instances indicates that a code of a set of codes was detected within noisy assessment data corresponding to a subject, wherein the noisy assessment data includes content originating from a care provider that identifies a characteristic of the subject or of a treatment for the subject, and wherein the subject has been diagnosed with multiple sclerosis;
inferring a type of multiple sclerosis that corresponds to the subject and the defined time period based on the set of code instances;
determining a temporal dynamic or distribution based on the set of code instances;
determining a modulation based on the temporal dynamic or the distribution;
determining whether an alert criterion is satisfied based on whether the modulation is above a threshold so as to represent noise or a predicted transition across types of multiple sclerosis;
outputting the inferred type of multiple sclerosis; and
when the alert criterion is satisfied:
generating an alert; and
outputting the alert.

12. The computer-implemented method of claim 11, further comprising:
selecting a treatment for the subject based on the inferred type of multiple sclerosis.

13. The method of claim 11, further comprising:
predicting that the subject is eligible for a clinical study based in part on the inferred type of multiple sclerosis.

14. The method of claim 11, further comprising:
assigning the subject to a cohort of a clinical study based at least in part on the inferred type of multiple sclerosis.

15. The computer-implemented method of claim 11, further comprising, in response to inferring that the type of multiple sclerosis corresponds to the subject and the defined time period:
inferring that the type of multiple sclerosis corresponds to the subject and another later time period that is subsequent to the defined time period.

16. The computer-implemented method of claim 11, wherein inferring that the type of multiple sclerosis corresponds to the subject and the defined time period includes:
detecting that the subject was inferred to have had another type of multiple sclerosis during another time period that was before the defined time period;
retrieving a type criterion associated with a previous inference of the other type of multiple sclerosis;
determining, based on the set of code instances, that the type criterion is not satisfied;
in response to determining that the type criterion is not satisfied, evaluating another type criterion using the set of code instances;
determining that the other type criterion is satisfied; and
determining that the type of multiple sclerosis is associated with the other type criterion.

17. The computer-implemented method of claim 11, wherein inferring that the type of multiple sclerosis corresponds to the subject and the defined time period includes:
detecting that the subject was inferred to have had another type of multiple sclerosis during another time period that was before the defined time period;
retrieving a type criterion associated with a previous inference of the other type of multiple sclerosis;
determining, based on the set of code instances, that the type criterion is not satisfied;
in response to determining that the type criterion is not satisfied, iteratively evaluating each of multiple other type criteria until one of the multiple other type criteria is satisfied; and
determining that the type of multiple sclerosis is associated with the one of the multiple other type criteria that is satisfied.

18. The computer-implemented method of claim 11, wherein the alert criterion is configured to be satisfied when the modulation indicates that the subject transitioned from a particular type of multiple sclerosis to a different particular type of multiple sclerosis, and wherein the transition is inconsistent with a definition of the different particular type of multiple sclerosis.

19. The computer-implemented method of claim 11, wherein the alert criterion is configured to be satisfied when the set code instances include a particular code instance representing a particular type of multiple sclerosis and a term representing an observation that is inconsistent with the particular type of multiple sclerosis.

20. A computer-program product tangibly embodied in a non-transitory machine-readable storage medium, including instructions configured to cause one or more data processors to perform a set of actions including:
selecting a set of code instances within a defined time period, wherein each of the set of code instances indicates that a code of a set of codes was detected within noisy assessment data corresponding to a subject, wherein the noisy assessment data includes content originating from a care provider that identifies a characteristic of the subject or of a treatment for the subject, and wherein the subject has been diagnosed with multiple sclerosis;
inferring a type of multiple sclerosis that corresponds to the subject and the defined time period based on the set of code instances;
determining a temporal dynamic or distribution based on the set of code instances;
determining a modulation based on the temporal dynamic or the distribution;
determining whether an alert criterion is satisfied based on whether the modulation is above a threshold so as to represent noise or a predicted transition across types of multiple sclerosis;
outputting the inferred type of multiple sclerosis; and
when the alert criterion is satisfied:
generating an alert; and
outputting the alert.
